(19) United States
(12) Patent Application Publication
(10) Pub. No.: US 2011/0288625 A1
(43) Pub. Date: Nov. 24, 2011

(54) Vessel Support Device and Methods for Supporting a Vessel

(75) Inventors: Michael K. Morgan, New South Wales (AU); Andrew S. Davidson, New South Wales (AU); Mike Skalsky, Sydney (AU)

(73) Assignee: MACQUARIE UNIVERSITY, New South Wales (AU)

(21) Appl. No.: 13/125,762

(22) PCT Filed: Oct. 29, 2009
(86) PCT No.: PCT/AU09/01415

§ 371 Date: Aug. 4, 2011

(30) Foreign Application Priority Data
Oct. 30, 2008 (AU) 2008905599
Oct. 29, 2009 (AU) PCT/AU2009/001415

(51) Int. Cl. A61F 2/84; A61F 2/82

(52) U.S. Cl. 623/1.11; 623/1.15; 623/1.18

(57) ABSTRACT

The present invention is directed to a vessel support member having opposing ends adapted to move in a circumferential plane, the support member configured for deployment in a substantially radial direction to support a substantial portion of the circumference of a vessel, the support member adapted to change between a first configuration and at least a second configuration, such that when in the first configuration, the support member is substantially linear or substantially planar, and as the support member is deployed and changes to at least the second configuration, one opposing end moves in a radial direction relative to the other opposing end, such that when in the second configuration the support member is substantially non-linear or substantially prismatic with a curved portion or region. Also provided is a method of supporting, and a kit for providing support to, a human or animal vessel, and a deployment means for deploying a support member.

### TECHNICAL FIELD

The present invention relates to devices and methods for supporting a vessel. More specifically, the present invention relates to devices and methods for supporting a vessel in the human or animal body, such as a vessel from the vasculature, biliary tract, gastrointestinal system or genito-urinary system.

### BACKGROUND

There are several medical reasons why it may be desirable or necessary to support a vessel in the body. Diseases and disorders of the vasculature resulting in, for example, arterial dilatation or stenosis can be associated with a high incidence of morbidity and, in several cases, mortality.

An arterial aneurysm is a dilatation of the artery. The dilatation may be asymmetrical (typically referred to as fusiform) or symmetrical (typically referred to as saccular). Dissecting aneurysms occur when blood splits the arterial media. Underlying causes of aneurysms include congenital, traumatic, mycotic, atherosclerotic and other causes. Most aneurysms in people from developed countries are caused by atherosclerosis.

Arterial aneurysms can occur in a range of regions of the body including thoraco-abdominal, abdominal, femoral, popliteal, visceral, carotid, subclavian and cerebral (for example, in the circle of Willis). Irrespective of size or location, all aneurysms have the potential to rupture.

Cerebral artery aneurysms occur in more than 2 percent of the population. Sixty-six percent of people in whom the aneurysm ruptures die.

Currently, there are three ways in which cerebral artery aneurysms are repaired to prevent their rupture. These are: craniotomy and clipping the aneurysm at the neck of the aneurysm (performed by a neurosurgeon); endovascular repair from within the lumen of the artery by coiling, stenting or both (performed in a radiology suite); and wrapping with material, such as muslin (performed by neurosurgeon).

Wrapping of cerebral artery aneurysms has had mixed success and is sometimes associated with complications, such as, optochiasmatic arachnoiditis (though rare). For this and other reasons, some authors have suggested that muslin wrapping of aneurysms should probably be avoided in aneurysms near the optic apparatus (see, for example, Taravati et al (2006) “That's a Wrap” 51(4) Survey of Ophthalmology July to August, 434 to 444). In any event, as a general proposition, current methods of wrapping are clumsy and possibly ineffective.

The endovascular repair may include stent deployment to hold detachable coils within the aneurysm lumen or sac. Although clipping and coiling are the preferred method of treating aneurysms, some aneurysms with a broad neck and/or with perforating arteries originating from the fundus, cannot be treated well by such techniques.

Currently, some of the typical ways of repairing a stenotic lesion are balloon angioplasty, stent deployment, or surgical by-pass grafts. The former, balloon angioplasty and stent deployment, are typically less invasive, only requiring access to the vasculature from a distal location, such as through a puncture site in the femoral artery. The latter, surgical by-pass grafts, are typically reserved for more severe or recurrent cases of stenosis and can, depending on the circumstances, be significantly more invasive than interventions involving balloon angioplasty and intravascular stent deployment.

Stents are typically cylindrical in shape and are transformable between a first cross-sectional diameter and a second, relatively larger, cross-sectional diameter. Currently, two different types of intravascular stents are used—balloon expandable stents, and self-expanding stents. During stent deployment interventions, a stent is introduced into the vasculature until adjacent the stenotic lesion. Once appropriately positioned, the stent is expanded, or allowed to expand, until its outer wall forces the stenotic lesion to open, and patency of the vessel is restored close to normal. The stent is typically left in vivo.

While stents provide a useful mechanism for dealing with stenoses, they have some drawbacks and, in some circumstances, lack suitable adaptability for certain anatomical sites, such as, for example, those dense with tortuous vasculature and those adjacent vessel bifurcations, among others.

The present inventors have developed devices and methods which provide an alternative and advantages over currently known devices and techniques for supporting a vessel.

### SUMMARY OF THE INVENTION

According to a first aspect, the present invention provides a vessel support member having opposing ends adapted to move in a circumferential plane, the support member configured for deployment in a substantially radial direction to support a substantial portion of the circumference of a vessel.

Preferably, the circumferential plane in which the opposing ends are adapted to move has (or if adapted to move in more than one circumferential plane, have) a different sized radius to the circumference of an interior wall of the vessel, an exterior wall of the vessel, or an interior or exterior wall of a vessel with an abnormality.

In one preferred embodiment, the support member is adapted to support at least a portion of an exterior wall of the vessel.

In another preferred embodiment, the support member is adapted to support at least a portion of an interior wall of the vessel.

In some particularly preferred embodiments, the support member is adapted to support a substantial portion of, or the whole of, the circumference of the vessel, whether from adjacent an inner wall or an external wall of the vessel. In some such preferred embodiments, the support member is adapted to support a substantial portion of, or the whole of, the circumference of the vessel and any variation to the circumference of the vessel, whether from adjacent an inner wall or an external wall of the vessel. In some such embodiments, the variation to the circumference of the vessel is provided by a dilatation or narrowing of a portion of the vessel wall or of the entire circumference of the vessel wall.

Typically, the vessel is from the vasculature, such as an artery or vein, although it may be any other vessel in the body, for example, a vessel in the biliary tract, the gastrointestinal system (including small or large bowel) or the genito-urinary system (including ureter or urethra). However, the vessel is not limited to a vessel from these tracts or systems, and may include all tubular body parts having vessel-like characteristics. Although this description focuses on uses of the invention with vessels from the vasculature, persons skilled in the art would appreciate that the invention can be used with other such vessels in a human or animal body.

The support member according to the present invention is particularly suitable for a cerebral artery.

Preferably, the support member is adapted to change, or be changed, between a first configuration and at least a second configuration. In a first configuration, the support member is preferably substantially linear. In a second configuration the support member is preferably substantially non-linear.

When in the first configuration, a body section of the support member interposed between the opposing ends is preferably substantially linear, although the body section may have slight curves, kinks or twists. Preferably, however, when in the first configuration, the support member is generally, or substantially, linear in spite of such curves, kinks, twists or other similar such non-linear variations.

When in the second configuration, a body section of the support member interposed between the opposing ends is preferably substantially non-linear, although the body section may have some substantially linear portions and additionally may have slight curves, kinks or twists. Preferably, however, when in the second configuration, the support member is generally, or substantially, non-linear in spite of such substantially linear portions, curves, kinks, twists or other similar such variations. In one particularly preferred embodiment, a cross-section of the support member in the second configuration has a substantially C-shape or is substantially circular.

The body section of preferred embodiments of the support member may be solid or may have weaker or weakened portions or perforations. In some preferred embodiments, the body section has a cross-hatched pattern or is mesh-like and may be formed by a plurality of intersecting strips or substantially linear segments, with weaker or weakened portions or perforations interposed between the intersecting strips or substantially linear portions. The weaker or weakened portions or perforations may be any shape and, in embodiments wherein the body section has a cross-hatched pattern or is mesh-like, their shape may be defined by the intersecting strips or substantially linear segments.

Opposing ends of the support member are adapted to move in a circumferential plane. The opposing ends may move in the same or different circumferential planes to one another. This or these planes of movement facilitate/s, in some preferred embodiments, a substantially non-linear (for example, substantially C-shape or substantially circular) cross-sectional shape for the support member when in the second configuration.

While substantially C-shaped or substantially circular are preferred cross-sectional shapes for the support member in the second configuration, the invention envisages a range of other cross-sectional shapes for the support member when in the second configuration. These include a substantially oval shape or any other shape that would facilitate deployment of the support member in a substantially radial direction including a shape with linear portions, such as, a substantially square, substantially rectangular or substantially polygonal, shape and include rounded or curved corners.

In some embodiments wherein the opposing ends move in different (for example, adjacent) circumferential planes, a plan view (and/or cross-section) of the support member in the second configuration also has a substantially non-linear shape, including any of the abovementioned or other substantially non-linear shapes.

There may be any one of a number of intermediate configurations assumed by the support member as it changes, or is changed, between the first and second configuration. There may also be additional configurations following change into the second configuration. Alternatively, the first and/or second configurations may be different to those described above.

In yet still further preferred embodiments, the support member has a third configuration having a substantially tubular shape. This shape can be attained in a number of ways, including the following:

(a) from the first configuration, the support member extends substantially transversely thereby becoming substantially planar. At least one opposing end of the substantially planar shaped support member moves in a radial direction in the same or similar circumferential plane as the other opposing end until the opposing ends are in close proximity or in contact; or

(b) from the second configuration, the support member extends substantially perpendicularly to its cross-section until a substantially tubular shape is formed.

The substantially tubular shape could also be attained by a combination of those changes in shape described above or in other ways. For example, a first configuration for the support member may have a substantially planar shape, in which case, the first part of the manner described in paragraph (a) above is not required. Preferably, the substantially tubular shape for the support member attained according to this embodiment has a cross-section corresponding to the cross-sectional shape of the support member in the second configuration.

In some such embodiments, the transverse extension (as described for paragraph (a) above) or the perpendicular extension (as described for paragraph (b) above), occurs with expansion of a segment of the support member that had previously been compressed or folded in, for example, a substantially concertina fashion. In the compressed or folded shape, prior to deployment, lateral and circumferential compression will be in variable ranges to suite the type of support member to be deployed and/or the geometry of the deployment means.

In some preferred embodiments, joining means join two or a plurality of support members together such that their body sections are adjacent and/or substantially parallel to each other. According to some such embodiments, each support member forms a rib-like component of the resultant greater sized support member. The rib-like components of some such embodiments provide the change from the first configuration to the second configuration, while the joining means provide the change to the substantially tubular configuration. Persons skilled in the art will readily appreciate that a support member itself can be formed of a plurality of such rib-like components so joined, or that two or a plurality of support members can be so joined giving rise to the above described “greater sized support member”.

In some such preferred embodiments, the joining means are expandable or extendible, including, for example, by being formed as a spring-like structure which may extend from a compressed state.

In yet still further preferred embodiments, the first configuration and at least the second configuration of the support member are both substantially non-linear. In some such preferred embodiments, cross-sections of the first and second configurations are similar or substantially identical to one another, including or excluding their relative sizes. For example, cross-sections of the first and second configurations are C-shaped or substantially circular, and the first configuration has a smaller, larger or substantially identical radius to that of the second configuration. Preferably, for embodiments wherein cross-sections of the first configuration and second configurations are C-shaped or substantially circular, a radius of the cross-section of the first configuration is smaller or larger than a radius of the cross-section of the second configuration. Such embodiments are particularly preferable for circumstances when the support member is being deployed intravascularly (though they may also be used when the support member is being deployed extravascularly). Suitable modes of deployment are described below in more detail.

In alternative embodiments wherein the first configuration and at least the second configuration of the support member are both substantially non-linear, cross-sections of the first and second configurations are different to one another.

In several preferred embodiments of the invention, particularly those wherein one configuration for a support member has a substantially tubular shape, the support member is adapted for preferential regulation of change between the first and second configurations and change to the substantially tubular shape configuration. In some such embodiments, for example, the support member is adapted so that the change from the first configuration to the second configuration occurs entirely, or almost entirely, before any change to the substantially tubular shape configuration. In other examples of such embodiments, the rates of these two changes are different to one another and the changes occur substantially simultaneously. In yet still further examples, the rates of the changes are the same or similar to each other, but they occur at different times, whether those times are overlapping or not.

According to some preferred embodiments, the ability for the support member to preferentially regulate changes between configurations is provided by virtue of the material or materials from which the support member is formed.

The support member is preferably formed of a material having shape memory properties. Such shape memory properties enable the support member to change between the first configuration and at least the second configuration. Preferably, the support member is formed so as to be biased into the second configuration. The support member of such embodiments can be manipulated into the first configuration and be maintained in that configuration by, for example, confinement, as when loaded or pre-loaded into deployment means. As the support member is deployed from the deployment means it assumes the second configuration having a bias to do so.

In a particularly preferred embodiment, the shape memory material is Nitinol. However, the invention envisages a wide range of other materials having shape memory properties. In some preferred embodiments, the support member is formed of a material having temperature-dependent martensitic phase transformation properties. In some such embodiments, these properties allow transformation from a low-symmetry to a highly symmetric crystallographic structure.

According to some preferred embodiments, the support member is formed of two or more materials or of one or more composite materials. In some such embodiments, for example, the body section and/or opposing ends of the support member are formed of a shape memory material, and connected or secured to a malleable or cloth-like material having biocompatible properties. Examples of such malleable or cloth-like materials include Dacron, Teflon, or any suitable biocompatible materials. The connecting or securing of, for example, the shape memory material to the malleable or cloth-like material can be achieved by stitching or any other suitable means.

By forming the support member of, for example, a shape memory material connected or secured to a malleable or cloth-like material, it is possible, in some embodiments, to reduce the overall rigidity of the support member, while increasing its flexibility and maintaining its strength. However, persons skilled in the art would appreciate that these features may also be achieved through other combinations of materials and/or with composite materials.

As noted above, use of different materials and/or composite materials in forming the support member may also assist in enabling preferential regulation of changes between configurations for the support member. In some preferred embodiments, for example, those wherein joining means join two or a plurality of support members together, or wherein a support member is formed of a plurality of rib-like components so joined, the joining means may be formed of material having first shape memory properties and the support members or rib-like components may be formed of another material having second shape memory properties.

In some such embodiments, the differences between the first shape memory properties and the second shape memory properties enables the preferential regulation of changes between configurations, such that one change in configuration can occur at a different time or rate to another change in configuration. For example, the first shape memory properties may have a different “time constant” to the second shape memory properties, such that expansion or extension of the joining means (and, therefore, change to the substantially tubular shape configuration) occurs more quickly than the change from the first substantially linear or planar configuration to the second substantially non-linear configuration.

Preferably, the opposing ends are maintained in close proximity or in contact (including overlapping each other) in the second configuration, or in the third or further configuration. In one preferred embodiment, the physical and/or mechanical properties of the support member, including, for example, the shape memory properties of a material from which a support member is formed, assist in the maintenance of the opposing ends in close proximity or in contact. In another embodiment, the opposing ends are bound together, such as, for example by twisting the opposing ends in an entwined fashion.

In some preferred embodiments, the opposing ends are maintained in close proximity or in contact by clamping means. Preferably, the clamping means is a clip, peg, tie or any other means capable of maintaining the opposing ends in close proximity or in contact.

In yet still further preferred embodiments, the opposing ends are maintained in close proximity or in contact in the second configuration by a combination of the above mechanisms. For example, the physical and/or mechanical properties of the support member may assist in the maintenance of the opposing ends as such, and to provide further additional assistance, a clamping means is also used.

In some preferred embodiments, at least one opposing end includes a tail member. The tail member may be substantially linear or substantially curved and may have a tail surface which corresponds with at least a clamping means surface on the clamping means. The corresponding tail surface and clamping means surface may have variegated contours or may have any corresponding contours that enable inter-engagement of the surfaces when in contact with each other. In such embodiments, the clamping means is preferably positioned so that its clamping means surface/s respectively inter-engage/s with the corresponding tail surface/s on the tail members.

The clamping means therefore maintains the opposing ends in close proximity or in contact and is inhibited from disengaging from the tail members by virtue of inter-engagement between corresponding tail surface/s and clamping means surface/s.

In other preferred embodiments, the tail surfaces of respective tail members may be corresponding such that the tail members themselves can inter-engage and maintain the opposing ends in contact or in close proximity. Also, in embodiments wherein only one opposing end includes a tail member, the tail surface may be corresponding to an end surface on the other opposing end, such that inter-engagement of the tail surface with the end surface can maintain the opposing ends in contact or in close proximity.

Persons skilled in the art will appreciate that the tail member need not have a tail surface specifically adapted for engagement or inter-engagement with another tail member, tail surface, corresponding clamping means surface or any other similar such member, means or surface. In some preferred embodiments, each opposing end has a tail member and the opposing ends are preferably maintained in contact or in close proximity by the mechanical or structural properties of the support member and/or by twisting the tail members around each other. In other preferred embodiments wherein each opposing end has a tail member, the opposing ends are preferably maintained in contact or in close proximity by the mechanical or structural properties of the support member and/or by clamping means.

In yet still further preferred embodiments wherein one, both or neither opposing end includes a tail member, the opposing ends may be maintained in close proximity or in contact additionally or alternatively by suturing or by other means. The invention envisages a range of means or mechanisms for maintaining the opposing ends in contact or in close proximity.

According to a second aspect, the present invention provides a vessel support member having opposing ends adapted to move in a circumferential plane, the support member configured for deployment in a substantially radial direction to support a substantial portion of the circumference of a vessel, the support member adapted to be changeable between a first configuration and at least a second configuration, such that when in the first configuration, the support member is substantially linear or substantially planar, and as the support member is deployed and changes or is changed to at least the second configuration, one opposing end moves in a radial direction relative to the other opposing end, such that when in the second configuration the support member is substantially non-linear or substantially prismatic with a curved portion or region.

According to a third aspect, the present invention provides a vessel support member having opposing ends adapted to move in a circumferential plane, the support member configured for deployment in a substantially radial direction to support a substantial portion of the circumference of a vessel, the support member adapted to change between a first configuration and at least a second configuration, such that when in the first configuration, the support member is substantially linear or substantially planar, and as the support member is deployed and changes to at least the second configuration, one opposing end moves in a radial direction relative to the other opposing end, such that when in the second configuration the support member is substantially non-linear or substantially prismatic with a curved portion or region.

According to a fourth aspect, the present invention provides a vessel support member having opposing ends adapted to move in a circumferential plane, the support member configured for deployment in a radial direction to support a substantial portion of the circumference of a vessel, the support member adapted to be changeable between a first configuration and at least a second configuration, such that when in the first configuration, the support member has a first substantially non-linear shape or substantially prismatic shape with a curved portion or region, and as the support member is deployed and changes or is changed to at least the second configuration, one opposing end moves in a radial direction relative to the other opposing end, such that when in the second configuration the support member has a second substantially non-linear shape or substantially prismatic shape with a curved portion or region.

In preferred embodiments of the support member of the fourth aspect, the first substantially non-linear shape or substantially prismatic shape with a curved portion or region, is substantially identical to the second substantially non-linear shape or substantially prismatic shape with a curved portion or region. In some such preferred embodiments and in other preferred and alternative embodiments, a cross-section of the first substantially non-linear shape or substantially prismatic shape with a curved portion or region is larger or smaller than a cross-section of the second substantially non-linear shape or substantially prismatic shape with a curved portion or region.

As noted above, when the support member of preferred embodiments is in a substantially non-linear configuration (whether in the first, second, or further configuration), the cross-section of that configuration can be substantially C-shaped, substantially oval shaped, or any other suitable shape, including a shape with linear portions, such as, a substantially square, substantially rectangular or substantially polygonal, shape and include rounded or curved corners. Accordingly, throughout this specification references to substantially “prismatic”, can encompass three-dimensional forms corresponding to each such cross-section. In some preferred embodiments, the substantially non-linear or substantially prismatic shape can therefore be substantially cylindrical, substantially tubular, and in some embodiments, may have some planar portions. In this specification, the term “prismatic” does not exclude substantially cylindrical, tubular or like shapes. According to a fifth aspect, the present invention provides deployment means adapted for deploying a support member according to the first, second, third or fourth aspects of the present invention, the deployment means comprising:

a substantially hollow member adapted to deliver the support member; and

an ejection means adapted to aid in the deployment of the support member from the substantially hollow member.

In some preferred embodiments, the substantially hollow member is defined by a substantially tubular wall. The substantially hollow member of some such embodiments, preferably those adapted for deploying a support member according to the fourth aspect of the invention, are open at at least one end. The substantially hollow member of some other such embodiments, particularly those adapted for deploying the support member according to the first, second, third or fourth aspects of the invention, are open at both ends. Preferably, the substantially hollow member is a catheter.

The substantially hollow member of some preferred embodiments includes at least one aperture extending through the substantially tubular wall. The aperture preferably provides fluid communication between at least a portion of the hollow and a space outside the substantially hollow member. In some embodiments, the aperture extends from and is part of one or both open ends of the substantially hollow member.

In some preferred embodiments, the support member is deployed from within the hollow of the substantially hollow member through the aperture. Preferably, the aperture has an aperture width, an aperture length and an aperture depth. The aperture width, aperture length and aperture depth may be consistent or may vary along their respective axes.

In some such preferred embodiments, the aperture has a first aperture width adjacent one side of the aperture depth, and a second aperture width adjacent an opposing side of the aperture depth. Preferably, the first aperture width is the width of the aperture penetrating an outer surface of the substantially tubular wall, and the second aperture width is the width of the aperture penetrating an inner surface of the substantially tubular wall. Preferably, the first aperture width is greater in size than the second aperture width.

In some such preferred embodiments, the aperture is defined on opposing sides along the aperture length by first and second aperture side walls, with each aperture side wall having an aperture side wall axis. In a particularly preferred embodiment, the aperture side wall axes are substantially unparallel. Preferably, the first aperture side wall extends tangentially from the inner surface of the substantially tubular wall until it meets the outer surface of the substantially tubular wall. In one such preferred embodiment, the second aperture side wall also extends tangentially from the inner surface of the substantially tubular wall until it meets the outer surface of the substantially tubular wall. Typically, this configuration for the first and second aperture side walls results in the aperture width being greater at its opening on the inner surface of the substantially tubular wall than the aperture width at its opening on the outer surface of the substantially tubular wall.

In other preferred embodiments, the second aperture side wall preferably extends at an acute angle to the inner surface of the substantially tubular wall in a direction away from the first aperture side wall until it meets the outer surface of the substantially tubular wall. Typically, this configuration for the first and second aperture side walls results in the aperture width being greater at its opening on the outer surface of the substantially tubular wall than the aperture width at its opening on the inner surface of the substantially tubular wall.

Persons skilled in the art will appreciate that one or both aperture side walls may extend tangentially from, at an acute angle to or at an obtuse angle to, the inner surface of the substantially tubular wall until it or they respectively meet the outer surface of the substantially tubular wall. In further alternative embodiments, the aperture side wall axes my be substantially parallel.

The aperture length may be smaller than or substantially equal to the aperture width and/or aperture depth. In some preferred embodiments, the aperture length is greater than the aperture width and/or aperture depth. Preferably the aperture length, aperture depth and aperture width are of a size adapted to at least accommodate a support member, or a cross-, longitudinal-, or other, section of the support member, passing through the aperture. A longitudinal axis of the aperture length is, in some such preferred embodiments, substantially parallel to a longitudinal axis of the substantially tubular wall.

Persons skilled in the art would appreciate that there are a range of dimensions, cross-sectional shapes, longitudinal-sectional and other sectional, shapes, positions and orientations for the aperture and that the configuration of these characteristics for a particular aperture may depend on a number of factors including, support member geometry, support member configurations, and preferred mode of deployment for a particular support member, among others. Persons skilled in the art would appreciate such preferred aperture characteristics suitable for different types of support member.

The ejection means of some preferred embodiments of the fifth aspect is an elongate member adapted to be inserted into the substantially hollow member. In some preferred embodiments, the elongate member is inserted into the substantially hollow member proximal the support member. For embodiments, wherein the support member is deployed from an open end of the substantially hollow member, the elongate member is advanced relative to the substantially hollow member (or the hollow member is retracted relative to the elongate member) until a leading end of the elongate member abuts a trailing end of the support member. Further relative advance of the elongate member (or relative retraction of the substantially hollow member) results in deployment of the support member.

The ejection means of some preferred embodiments, particularly those adapted for aiding in the deployment of a support member according to the fourth aspect, include guide means for guiding the support member through deployment.

In some such embodiments, the guide means is a guide member extending from a leading end of the elongate member. In some preferred configurations, the guide member extends from an outer edge of the leading end of the elongate member. In such embodiments, the guide member is adapted to locate between opposing ends of the support member when the support member is in the first configuration. These particular embodiments of the guide member are well suited to assist in the deployment of a support member which, when in the first configuration, has a substantially circular or C-shaped cross-section.

As the guide member locates between opposing ends of the support member, the guide member effectively (though not necessarily completely) “fills a gap” between opposing ends of the support member. In this way, a cross-section of the support member with the guide member located as such is effectively a complete or almost complete circle. In alternative embodiments, although the guide member locates between opposing ends of the support member it need not “fill the gap” between them, but in cross-sectional view, the guide member “adds” to the incomplete circle provided by the support member.

In some such embodiments, a side of the guide member can abut a trailing end of the support member. The support member can then be deployed through the aperture in the substantially tubular wall of the substantially hollow member by relative rotation between the ejection means and the substantially hollow member.

In other preferred embodiments of the deployment means according to the fifth aspect, particularly those adapted for deploying the support member through an end opening of the substantially hollow member, the substantially hollow member may not require an aperture in the substantially tubular wall. Preferably, the ejection means of such embodiments include an elongate member adapted to be loaded or preloaded into the substantially hollow member proximal the support member. Preferably, deployment means according to such embodiments are adapted so that advancement of the elongate member, or retraction of the substantially hollow member, relative to the support member causes deployment of the support member through the opening at the leading end of the substantially hollow member.

The ejection means may also be appropriately preloaded into the substantially hollow member, or may form part of, or be operably connected to, the substantially hollow member. Similarly, the support member may be preloaded into the substantially hollow member.

In some preferred embodiments, a trailing end or other suitable part of the support member is joined to the ejection means. Suitable joins can be selected from the group consisting of frangible joins, snap-off joins, tearable joins, breakable joins, detachable joins and a combination of two or more thereof. In some preferred embodiments, the join is an electrolytically detachable join. In some such embodiments, when an electric current is passed through the ejection means and/or the support member, the electrolytic join dissolves releasing the support member. The invention envisages other types of joins and associated means of releasing those joins. In other preferred embodiments, there is no physical join between the ejection means and the support member.

In yet still further preferred embodiments, the deployment means of the fifth aspect of the present invention further includes an introduction catheter adapted to receive the substantially hollow member. The introduction catheter of preferred embodiments is preferably adapted to provide access to the deployment site. The access provided is preferably used by the substantially hollow member and may also be used by other instruments or equipment that could aid in deployment, positioning or orientation of the support member as required.

According to a sixth aspect, the present invention provides a kit for providing support to a vessel in a human or animal body, the kit comprising:

a support member according to the first, second, third or fourth aspects of the present invention; and deployment means according to the fifth aspect of the present invention.

In some preferred embodiments, the kit further includes at least one probe-like structure adapted to assist in determining requisite support member geometry, Preferred embodiments of the probe-like structure are described below with reference to the seventh aspect of the invention.

According to a seventh aspect, the present invention provides a method of supporting a human or animal vessel comprising:

deploying a vessel support member according to the first, second, third or fourth aspects of the present invention at, or in the vicinity of, a deployment site such that the support member substantially contours a portion of a circumference of the vessel, thereby supporting the vessel.

As noted above, the human or animal vessel may be any vessel from the human or animal body including, for example, a vessel from the vasculature, the biliary tract, the gastrointestinal system (including small or large bowel), the genito-urinary system (jncluding ureter or urethra) or it may be any tubular body part having vessel-like characteristics.

For the purpose of illustration, the description now focuses on preferred embodiments of the method, particularly as applicable to deployment of a support member for supporting a blood vessel, and more preferably an artery.

Preferably, the deployment site is within, or adjacent, substantially the same or similar circumferential plane as a portion of the vessel which is abnormal. In one preferred embodiment, the abnormal portion of vessel includes a stenosis, dilatation or other aneurysm.

The support member can be deployed intra- or extra-vascularly. Typically, to support a stenotic vessel, the support member is deployed intra-vascularly and to support a vessel with a dilatation or aneurysm, the support member is deployed intra- or extra-vascularly. Preferred modes of deployment of the support member according to the present invention are various and may depend on whether the support member is deployed intra- or extra-vascularly, the support member geometry, and the shape or shapes of the support member in the first, second and other configurations, among other factors determinable by persons skilled in the art.

The method of preferred embodiments, whether involving intra- or extra-vascular deployment, may be carried out through an open operative wound or may be carried out using minimally invasive techniques, including, for example, endoscopy or laparoscopy.

In a preferred embodiment, the method may include a number of initial steps to be undertaken prior to deployment of the support member. In one preferred initial step a deployment site relative to the vessel to be supported is identified. This identification can be done with radiographic guidance or by other suitable means or mechanisms.

In another preferred initial step of the method, a determination of requisite support member geometry is taken.

In one preferred embodiment, a probe-like structure having variable physical characteristics, including, for example, variable lengths, angles and/or terminal curves, adapted to correspond to relevantly corresponding physical characteristics of the support member in the first, second or other configuration, is advanced into the deployment site. The probe-like structure of this embodiment preferably alters, or is altered, in physical configuration until it substantially matches the requisite support member geometry for supporting the vessel or the abnormal portion of vessel.

In other preferred embodiments, a plurality of probe-like structures with substantially fixed physical characteristics, adapted to correspond to relevantly corresponding physical characteristics of support members of varying physical characteristics. are advanced adjacent the deployment site one by one to assist in determining the requisite support member geometry. In one such embodiment, each probe-like structure includes a measuring member with an arc of curvature having a radius adapted to correspond to an arc of curvature on an inner or outer part of a vessel wall and/or an inner or outer part of an abnormality in a vessel wall, such as a stenosis or an aneurysm. Ultimately, the probe-like structure having a measuring member with an arc of curvature which most closely resembles the arc of curvature of part of the vessel wall and/or part of the abnormality in the vessel wall is determined.

The probe-like structure of either of the above described embodiments is then removed and used as a guide for selecting a suitable support member. The invention envisages other mechanisms or means for determining the requisite support member geometry.

In another preferred initial step a suitable support member is selected. In one preferred embodiment, the selected support member is suitable if it has the requisite support member geometry.

In yet another preferred initial step, a suitable deployment means is selected. Selection of the suitable deployment means can be undertaken in a number of ways. Whether a particular deployment means is suitable may depend on a number of factors including, but not limited to, the requisite support member geometry, the anatomical region into which the deployment means will be advanced and the desirable surgical approach including the proposed mode of deployment, whether from within or from outside the vessel.

In yet another preferred initial step, a support member is preloaded into the deployment means. Alternatively, the support member is introduced into the deployment means when a leading end of the deployment means is located adjacent a deployment site.

Persons skilled in the art will appreciate that one or more of the preferred initial steps mentioned above may not be necessary. Similarly, persons skilled in the art will appreciate that the preferred initial steps that are undertaken may be undertaken in any suitable order.

In some particularly preferred embodiments, the support member is deployed extra-vascularly. One preferred mode of deployment of one preferred support member to support an artery, for example, with an aneurysm is as follows: the support member is caused to adopt the first substantially linear (or planar) configuration and loaded into the deployment means. In preferred embodiments, the cross-sectional area of that part of the deployment means (and, in some embodiments, of that part of a substantially hollow member of the deployment means) in which the support member is loaded need only accommodate the cross-sectional area of the support member in the first substantially linear configuration (or planar).

In one preferred embodiment of this method of extravascular deployment, a leading end of the deployment means is advanced through body tissue until adjacent the deployment site. In embodiments wherein the deployment site is at or adjacent an aneurysm the leading end of the deployment means is advanced through body tissue until adjacent an apex or outer most portion of the aneurysm.

Preferably, an ejection means, such as a pushing rod, is inserted into the deployment means proximal the support member until it abuts a trailing end of the support member. The ejection means is then advanced, or the deployment means is retracted, relative to the support member, thereby deploying the support member. As the support member is deployed, one opposing end (the leading end) moves in a radial direction in the same or similar circumferential plane to the other opposing end (the trailing end) until such time as the support member is fully deployed and has assumed the second substantially non-linear (or prismatic) configuration. Preferably, when in the second substantially non-linear (or prismatic) configuration, the support member substantially encircles the vessel and the aneurysm. In particularly preferred embodiments, once fully deployed, the opposing ends of the support member are adjacent or abut opposing sides or walls of the aneurysm. In some such embodiments, when the opposing ends come in to close proximity to each other, the aneurysm is compressed between the opposing ends. In some such circumstances, this substantially obliterates or substantially eliminates the aneurysm.

In other preferred embodiments, the opposing ends come into close proximity or into contact with each other in such a way that when in the second substantially non-linear (or prismatic) configuration the support member substantially contains the vessel wall and the aneurysm.

In cases where the deployment site is in or adjacent extravascular space large enough to accommodate relatively large surgical tools and equipment, the support member can be deployed adjacent the deployment site. Once deployed, the support member assumes the second substantially non-linear (or prismatic) configuration and can be manipulated into the first substantially linear (or planar) configuration or an intermediate configuration between the second and first configurations until within the deployment site where it can be released so as to support the vessel as required or desired. In such cases, it may also be possible, given adequate extravascular space to similarly deploy a support member having first and at least second configurations that have the same or similar cross-sectional non-linear shapes, such as, substantially circular or C-shaped.

Other preferred modes of deployment can be used when the support member having first substantially linear (or planar) and second substantially non-linear (or prismatic) configurations is deployed intra-vascularly. In some such embodiments, the deployment means (loaded with the support member in the first substantially linear (or planar) configuration) is inserted into a peripheral vessel, such as the femoral artery, and advanced through the vasculature until appropriately positioned for deployment of the support member at or in the vicinity of the deployment site. Once so positioned, in one embodiment, the deployment means is manipulated so that a portion of its distal end curves so as to be advancing substantially perpendicular to its longitudinal axis and contouring a portion of the vessel wall. Preferably, the distal end is curved as such when in the same or similar circumferential plane as the deployment site. The support member is then deployed so as to support the vessel. Deployment of the support member may be effected by any suitable means including with the use of an ejection means as described above in relation to one extravascular deployment method.

As the support member is deployed, one opposing end (the leading end) moves in a radial direction in the same or similar circumferential plane to the other opposing end (the trailing end) until such time as the support member is fully deployed and has assumed the second substantially non-linear (or prismatic) configuration. Preferably, when in the second substantially non-linear (or prismatic) configuration, the support member substantially replicates a circumferential portion of the interior wall of the vessel. In some such circumstances, the support member preferably supports the patency of the vessel, and more preferably still, provides a substantial division between the lumen of the vessel and the aneurysmal sac.

In cases where the deployment site is in or adjacent extravascular space large enough to accommodate relatively large surgical tools and equipment, a support member having first and at least second configurations that have the same or similar cross-sectional non-linear (or prismatic) shapes may be deployed in a similar manner extra-vascularly.

In some embodiments where the distal end of the deployment means does not, or cannot be made to, curve as explained above, the support member is deployed, and once deployed and in the second substantially non-linear (or prismatic) configuration, is moved or adjusted into the preferred position and orientation (whether deployed intra- or extra-vascularly).

In other preferred embodiments of the method of intra-vascular deployment, a support member having first and at least second configurations that have the same or similar cross-sectional non-linear (or prismatic) shapes may be used. As described above, in one such embodiment, the non-linear cross-sectional shape is substantially circular or substantially C-shaped.

Yet still further preferred modes of deployment can be used when the support member having first substantially non-linear (or prismatic) and at least second substantially non-linear (or prismatic) configurations is deployed intra-vascularly. One such preferred mode of deployment is as follows: the support member is caused to adopt the first substantially non-linear (or prismatic) configuration and loaded or pre-loaded into the deployment means. In preferred embodiments, the cross-sectional area of that part of the deployment means (and, in some embodiments, of that part of a substantially hollow member of the deployment means) in which the support member is loaded need only accommodate the cross-sectional area of the support member in the first substantially non-linear configuration (or prismatic).

In one preferred embodiment of this method of intra-vascular deployment, a leading end of the deployment means is advanced through body tissue until adjacent the deployment site. Preferably, the deployment means includes a substantially hollow member defined by a substantially tubular wall with an aperture passing there through, the aperture having an aperture width, an aperture depth and an aperture length. The aperture width, aperture depth and aperture length are all adapted to accommodate a support member passing there through. Preferably, the aperture is positioned such that a longitudinal axis of the aperture length is substantially parallel to a longitudinal axis of the tubular wall. The deployment means for one preferred mode of deployment according to this embodiment further includes an ejection means including an elongate member and a guide member extending from a leading end of the elongate member at an outer edge of the elongate member.

Once access to the vasculature has been achieved, the substantially hollow member is advanced along the vasculature until the aperture is positioned adjacent the deployment site. The ejection means is then loaded or preloaded into the substantially hollow member proximal the support member such that the guide member is interposed between opposing ends of the support member. Preferably, prior to deployment, the support member is positioned so as to be in the same circumferential plane as the aperture.

Relative rotation between the ejection means and the substantially hollow member causes rotation of the support member until one end (the leading end) of the support member begins to pass through the aperture. As the support member is deployed, the leading end moves in a radial direction in the same or similar circumferential plane to the other opposing end (the trailing end) until such time as the support member is fully deployed and has assumed the second substantially non-linear (or prismatic) configuration. Preferably, when in the second substantially non-linear (or prismatic) configuration, the support member substantially contours an internal wall of the vessel and substantially bridges the neck of the aneurysm or the extent of a stenotic legion.

The support member may be deployed at or adjacent a vessel bifurcation, whether deployed intra- or extra-vascularly. This provides a further important advantage over currently known devices and methods for supporting a vessel which are generally deficient or complex in the context of providing vessel support at or near a bifurcation.

In yet still further preferred embodiments, the method of the present invention further includes maintaining the opposing ends of the support member in close proximity or in contact. Preferably the maintaining is achieved by applying clamping means to the opposing ends or by other means or mechanisms as described above in relation to the support member of the first aspect of the invention.

In yet still further preferred embodiments, the clamping means or other means or mechanisms for maintaining the opposing ends in close proximity or in contact can be removed after having been employed. In this way, adjustments can be made to the position and/or orientation of the support member or the support member can be removed.

As explained above, preferred embodiments of the deployment means may further include an introduction catheter adapted to create a channel through which deployment means loaded or preloaded with the support member can be passed. The introduction catheter and the deployment means may have the same or differently shaped cross-sections. In one preferred embodiment, both the introduction catheter and the deployment means have a substantially circular cross section. In another preferred embodiment, the introduction catheter has a circular cross-section and the deployment means has an elliptical cross-section.

In further preferred embodiments of the method of the seventh aspect of the present invention, whether applied intra- or extra-vascularly, a further step may be the introduction of a further or several further support members to the deployment site. The plurality of support members may be adjacent each other and collectively traverse the abnormal portion of the vessel or both abnormal and normal portions of the vessel. For some such embodiments, the support member as noted above which is formed of a plurality of rib-like components joined by joining means such that their body sections are adjacent and/or substantially parallel to each other, may be used. Alternatively, the plurality of support members may be joined together by such joining means also as noted above.

In further embodiments of the method, one or more support members are deployed intravascularly and one or more support members are deployed extravascularly. In one such embodiment, once so deployed, the vessel wall between the support members is preferably splinted or more supported than if a support member was deployed only intravascularly or only extravascularly. Preferably, the respective pressures which the intravascularly deployed support member and the extravascularly deployed support member apply to the vessel wall are adequate to support the vessel wall without jeopardizing its viability.

The method of the seventh aspect has been described as being carried out intra- or extra- vascularly and with particular reference to arterial deployment. As noted above, the support member of the present invention can also be used with other vessels in human or animal bodies. Persons skilled in the art would appreciate that the general principles of deployment discussed above are also applicable to internal or external deployment of the support member in relation to vessels outside the vasculature.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention.

In order that the present invention may be more clearly understood, preferred embodiments will be described with reference to the following drawings and examples.

### BRIEF DESCRIPTION OF THE DRAWINGS

FIG. 1A is a perspective view of one preferred embodiment of a support member when in a first configuration.

FIG. 1B is a perspective view of the support member depicted in FIG. 1A when in a second configuration.

FIG. 2 is one preferred embodiment of a support member.

FIGS. 3A to 3F are a series of cross-sectional views of various different embodiments of a support member.

FIGS. 4A to 4C are a series of perspective views of different embodiments of a support member, with each embodiment illustrating use of a different device or mechanism for maintaining opposing ends of the support member in close proximity to one another or in contact.

FIGS. 5A and 5B are schematic diagrams illustrating different embodiments of a deployment means.

FIGS. 6A and 6B are schematic diagrams illustrating different embodiments of a deployment means.

FIG. 7A is perspective view of an embodiment of a substantially hollow member from a deployment means of a preferred embodiment.

FIG. 7B is a perspective cut-off view of the substantially hollow member depicted in FIG. 7A as cut along line A-A, and FIG. 7C is a blown up view of the upper portion of one embodiment of an aperture as circled in FIG. 7B.

FIG. 7D is a perspective view of one embodiment of an ejection means from a deployment means.

FIG. 7E is a cross-sectional view of an embodiment of the deployment means having a substantially hollow member as depicted in FIG. 7A, ejection means as depicted in FIG. 7D and a support member loaded in the deployment means.

FIGS. 8A to 8D are schematic diagrams illustrating several embodiments of a measuring device, wherein each measuring device depicted has different fixed physically characteristics to the others.

FIG. 8E is a measuring device according to a preferred embodiment, wherein the measuring device has a plurality of physical characteristics which are variable.

FIG. 8F is a schematic illustration showing a measuring device being used to measure the physical characteristics of an artery with an aneurysm. As illustrated, the measuring device embodiment depicted in FIG. 8C appears to represent a measuring device having fixed physical characteristics which most closely correspond to the physical characteristics of the blood vessel with the aneurysm.

FIGS. 9A to 9C are progressive schematic illustrations depicting deployment of a support member according to one preferred embodiment by a deployment means according to one preferred embodiment to support a blood vessel with an aneurysm. The mode of deployment depicted is extra-vascular deployment.

FIG. 10A is a perspective illustration depicting a deployment means according to one preferred embodiment in vivo ready to deploy a support member of another preferred embodiment to support a vessel with an aneurysm. The mode of deployment depicted is extra-vascular deployment.

FIG. 10B illustrates a similar embodiment to that depicted in FIG. 10A, showing the support member being deployed.

FIG. 10C is a perspective illustration depicting a deployment means according to one preferred embodiment in vivo, having deployed a support member according a preferred embodiment of the invention so that it is supporting a blood vessel. The mode of deployment depicted is extra-vascular deployment.

FIGS. 11A and 11B are schematic illustration depicting progressive steps in the deployment of a support member according to one preferred embodiment being deployed by a deployment means according to one preferred embodiment in a vessel having an aneurysm. The mode of deployment depicted is intra-vascular deployment.

FIGS. 12A and 12B are schematic illustrations depicting progressive steps in the deployment of a support member according to one preferred embodiment being deployment by deployment means according to one preferred embodiment in a blood vessel having an aneurysm. The mode of deployment depicted is intra-vascular deployment.

FIG. 13A is schematic illustration of a support member according to one preferred embodiment being deployed by deployment means according to one preferred embodiment into a blood vessel having a stenotic lesion. The mode of deployment depicted is intra-vascular deployment.

FIG. 13B is a schematic illustration of the support member according to the embodiment depicted in FIG. 13A being deployed from the deployment means according to the embodiment depicted in FIG. 13A.

FIG. 13C is a perspective schematic illustration following on from FIG. 13B illustrating how relative rotation of the substantially hollow member with respect to the ejection means aids in the deployment of the support member. This figure also illustrates a support member already having been deployed according to this mode of deployment. Accordingly, this figure further illustrates how a plurality of support members can be deployed to support a vessel.

FIG. 14A is a front view of another preferred embodiment of a support member when in a first configuration. The body section of this embodiment has a cross-hatched pattern or is mesh-like.

FIGS. 14B and 14C are schematic illustrations depicting progressive steps in the deployment of a support member according to the embodiment depicted in FIG. 14A being deployed by a deployment means according to one preferred embodiment.

FIG. 14D is a front view of another preferred embodiment of a support member when in a first configuration. The support member is formed of more than one material and includes a malleable or cloth-like material having biocompatible properties. The body section of this embodiment has a cross-hatched pattern or is mesh-like.

FIG. 15A is a schematic illustration of an artery which bifurcates and a fusiform aneurysm adjacent the bifurcation.

FIG. 15B is a schematic illustration depicting two support members according to the embodiment depicted in FIG. 14A that have been deployed and are supporting the vessel and fusiform aneurysm depicted in FIG. 15A. The mode of deployment for the support members was extra-vascular deployment.

FIG. 16A is a front view of another preferred embodiment of a support member when in a first configuration. In this embodiment, rib-like components are provided by a plurality of support members. The rib-like components are joined by a plurality of joining means.

FIGS. 16B to 16D are schematic illustrations providing an example of progressive steps in which the embodiment of the support member depicted in FIG. 16A can be compressed for loading or preloading into deployment means. The support member depicted in these figures is formed of more than one material and includes a malleable or cloth-like material having biocompatible properties.

FIG. 16E is a schematic illustration depicting a support member according to the embodiment depicted in FIG. 16A that has been deployed and is supporting a vessel adjacent a bifurcation. The mode of deployment for the support member was extra-vascular deployment.

### DETAILED DESCRIPTION OF PREFERRED EMBODIMENTS

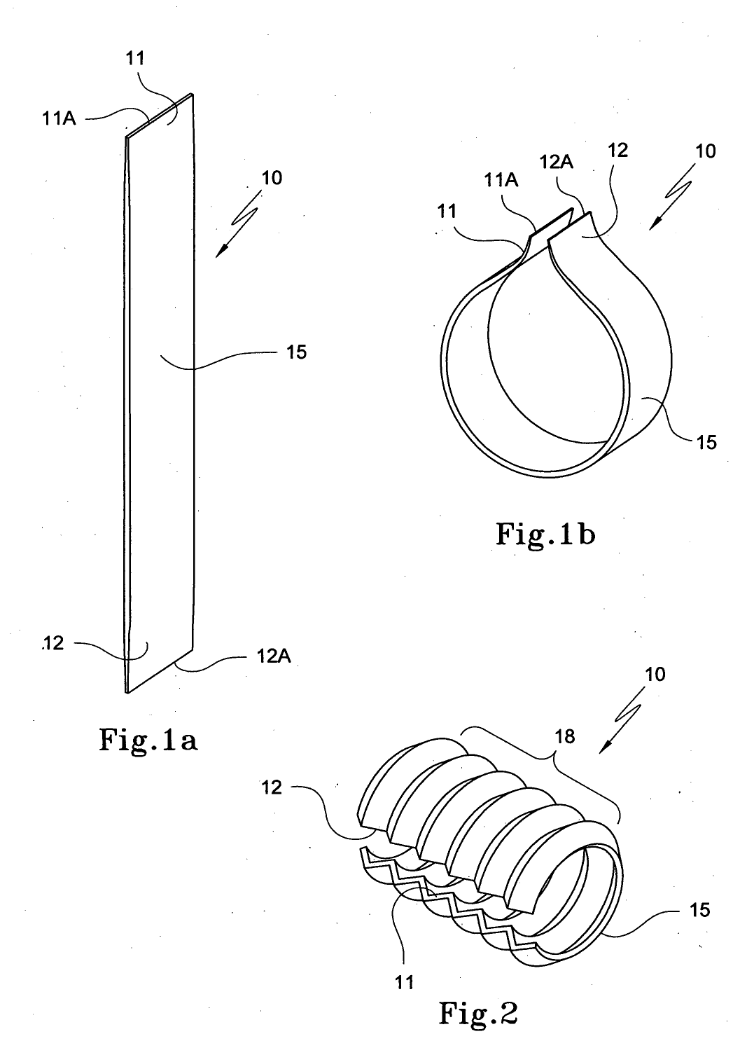

Various preferred embodiments of support member 10 according to the present invention are depicted variously throughout the figures and in particular detail are depicted in FIGS. 1 to 4, 14A, 14D and 16A to 16D.

Support member 10 has opposing ends 11 and 12 adapted to move in a circumferential plane. The support member 10 is configured for deployment in a substantially radial direction to support a substantial portion of the circumference of a vessel 50. Support member 10 can be used to support at least a portion of an exterior wall of the vessel 50 or at least a portion of an interior wall of the vessel 50. Preferably, support member 10 is adapted to support a substantial portion of, or the whole of, the circumference of vessel 50 whether from adjacent in an inner wall or an external wall of the vessel 50. In some preferred embodiments, supporting a substantial portion of, or the whole of, the circumference of vessel 50 includes supporting any variation of the circumference of the vessel, such as an aneurysm 51 or a stenosis 52.

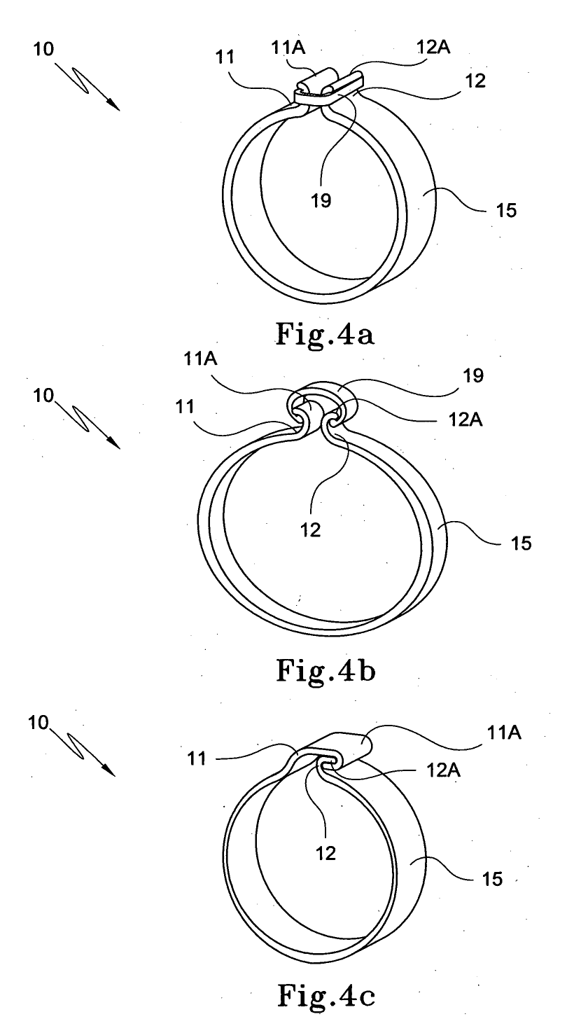

In preferred embodiments, support member 10 is adapted to change, or be changed, between a first configuration and at least a second configuration. FIGS. 1A, 1B and 3A depict various views of support member 10 according to one preferred embodiment. FIG. 1A illustrates support member 10 in a first preferred configuration wherein the support member 10 is preferably substantially linear or substantially planar. FIGS. 1A and 1B in particular illustrate body section 15 having a width and may therefore be regarded as substantially planar. Support members 10 according to other preferred embodiments may be formed such that body section 15 width is narrow, in which case the shape of the support member 10 in the first configuration is substantially linear.

As shown in FIG. 1B, when in the second configuration, the body section 15 of support member 10 is preferably substantially non-linear. In a particular preferred embodiment, a cross-section of this support member 10 in the second configuration has a substantially C-shape or is substantially circular.

Opposing ends 11 and 12 may move in the same or different circumferential planes to one another as support member 10 is deployed.

As is well illustrated in FIGS. 3A-3F, the invention envisages a range of cross-sectional shapes for the support member 10 when in the second configuration. These include substantially oval (see, for example, FIGS. 3B and 3D), and may include other shapes (not shown) that would facilitate deployment of support member 10 in a substantially radial direction.

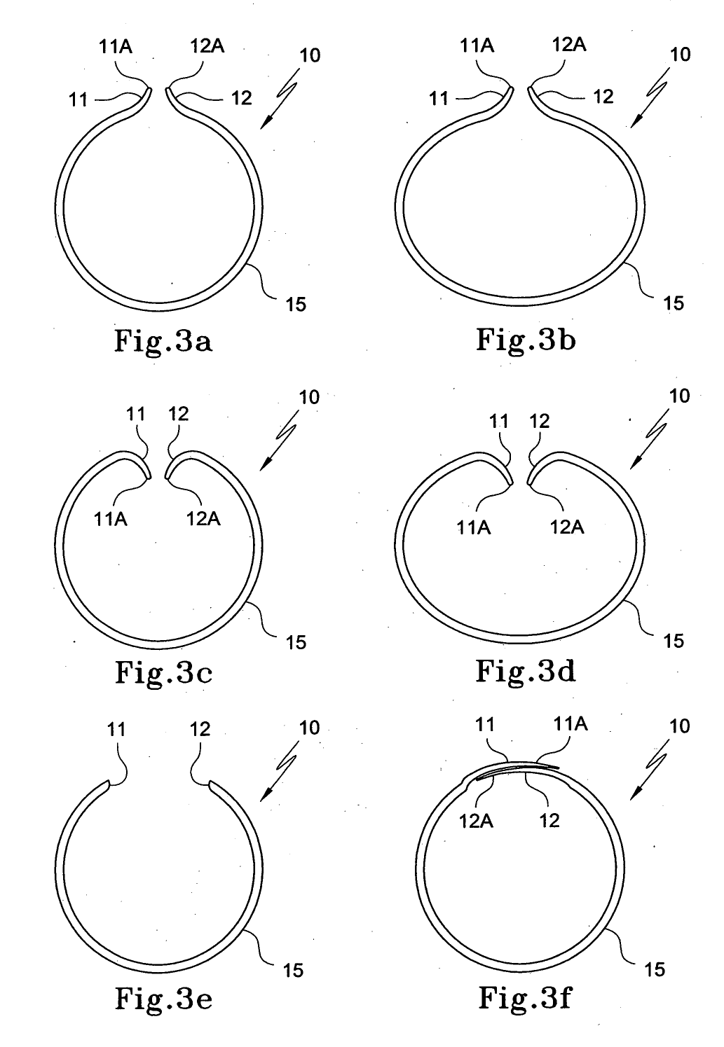

There may also be any one of a number of intermediate configurations assumed by support member 10 as it changes, or is changed, between the first and second configurations. For example, FIG. 2 illustrates a further preferred embodiment of support member 10 having a third configuration with a substantially tubular shape. The shape can be attained in a number of ways including:

(a) From the first configuration, support member 10 extends substantially transversely 18 thereby becoming substantially planar (that is increasing its width as discussed above). Then, at least one opposing end 11 of the substantially planar shaped support member 10 moves in a radial direction in the same or similar circumferential plane as the other opposing end 12 until the opposing ends 11 and 12 are in close proximity or in contact; or

(b) From the second configuration, the support member extends substantially perpendicularly 18 to its cross-section until a substantially tubular shape is formed.

In some such embodiments, transverse extension 18 (as describes paragraph (a) above) for perpendicular extension 18 (as described for paragraph (b) above), occurs with expansion of a segment of support member 10 that had been previously compressed or folded in a substantially concertina fashion. These proposed embodiments are well illustrated in FIG. 2. The invention envisages other mechanisms for transverse or perpendicular extension.

Further preferred embodiments of the support member 10 provide that the first configuration and at least the second configuration are substantially non-linear. In some such preferred embodiments, cross-sections of the first and second configurations are similar or substantially identical to one another, including or excluding their relative sizes. In one such embodiment, the support member 10 has a first substantially non-linear shape or substantially prismatic shape with a curved portion or region, and as the support member 10 is deployed and changes, or is changed, to at least the second configuration, one opposing end 11 moves in a radial direction relative to the other opposing end 12, such that when in the second configuration the support member 10 has a second substantially non-linear shape or substantially prismatic shape with a curved portion or region.

Support members 10 of preferred embodiments are formed from a material having shape memory properties, such as Nitinol. Preferably, support member 10 is formed so as to be biased into the second configuration. The invention also envisages a wide range of other materials having shape memory properties or temperature-dependant maternisitic phase transformation properties. In some preferred embodiments, the support member 10 is formed of at least two materials having shape memory properties, wherein each of the materials have at least one characteristic of the shape memory properties different to the other. For example, the shape memory properties of one of the materials may be faster at effecting changes in shape than the other.

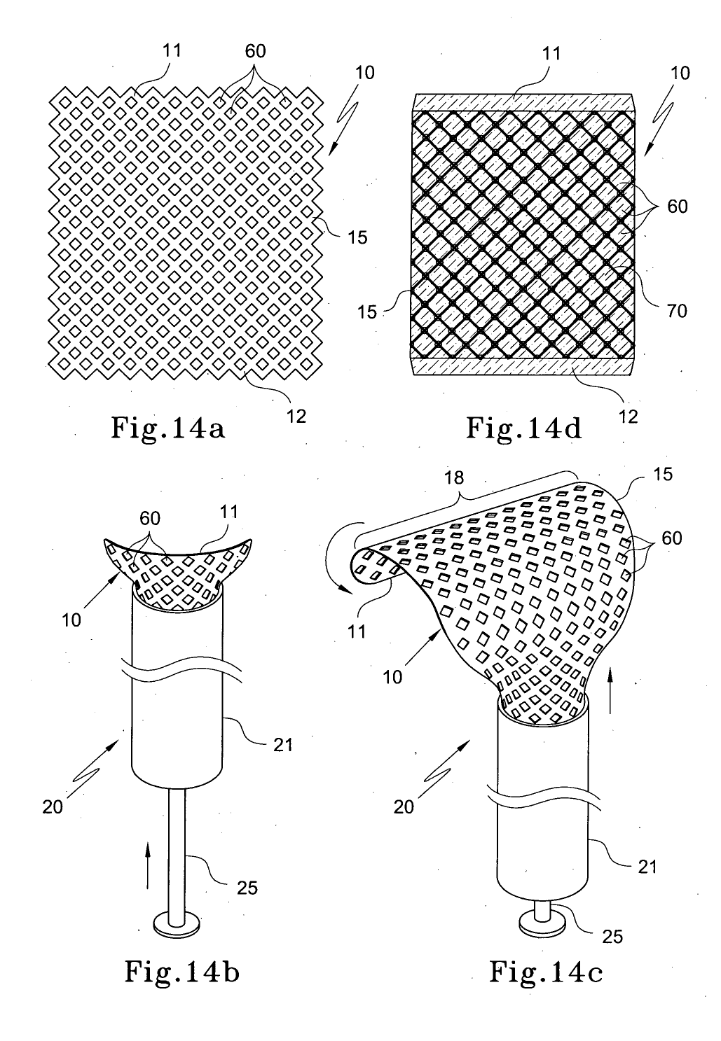

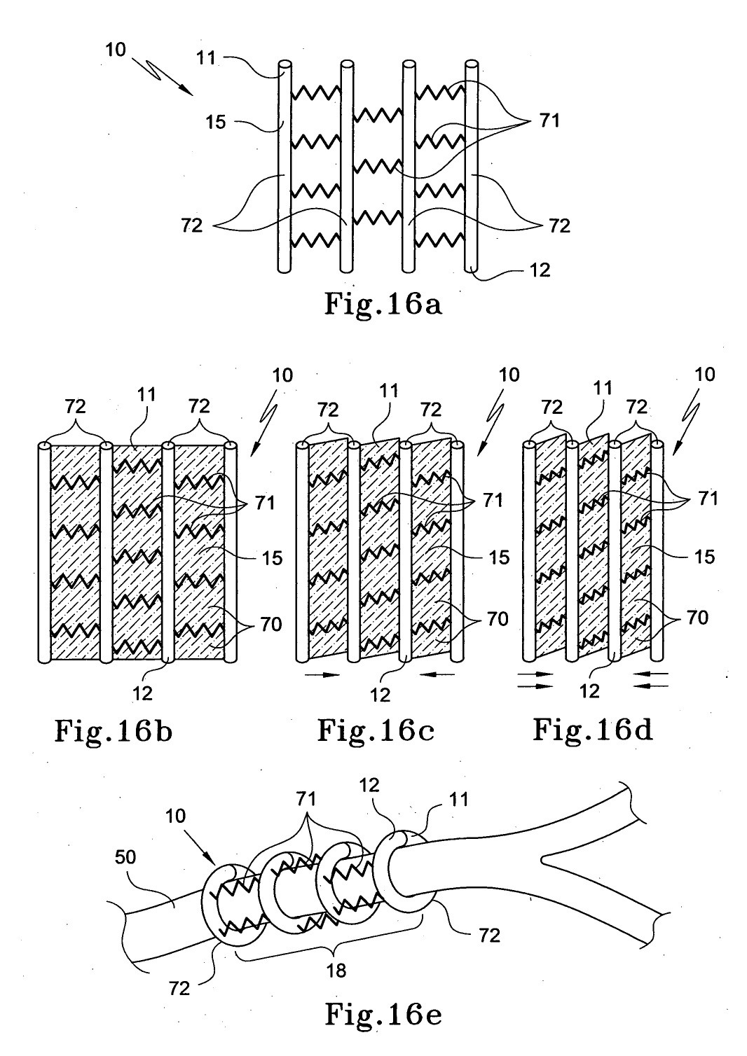

In yet still further preferred embodiments, support member 10 is formed of composite materials. In some preferred embodiments, support member 10 is formed of at least one material having shape memory properties and connected or secured to a malleable or cloth-like material 70 having biocompatible properties. Examples of such malleable or cloth-like materials 70 include Dacron, Teflon, or any suitable biocompatible materials. The connecting or securing of, for example, the shape memory material to the malleable or cloth-like material 70 can be achieved by stitching or any other suitable means. FIGS. 14D and 16B to 16D depict some preferred embodiments of support members 10 having a malleable or cloth-like material having biocompatible properties connected or secured to the material having shape memory properties.

As explained above, by forming the support member 10 of, for example, a shape memory material connected or secured to a malleable or cloth-like material 70, it is possible, in some embodiments, to reduce the overall rigidity of the support member 10, while increasing its flexibility and maintaining its strength. The embodiment depicted in FIG. 14D illustrates this well with relatively less material having shape memory properties being required in the formation of the support member 10 (as compared, for example to the embodiment of the support member 10 depicted in FIG. 14A).

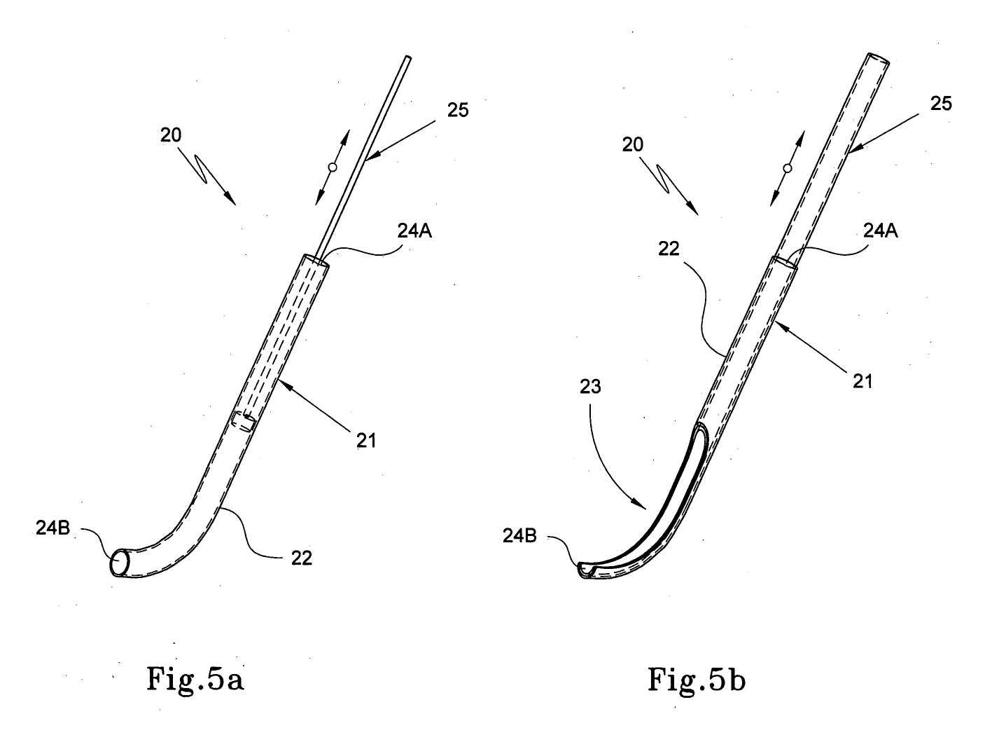

Opposing ends 11 and 12 are preferably maintained in close proximately or in contact once in the second configuration. In one embodiment, such maintenance is achieved as a result of the physical and/or mechanical properties of support member 10. In other embodiments, opposing ends 11 and 12 are bound together such as, for example, by use of a clamping means 19. FIGS. 4A and 4B illustrate different embodiments of clamping means 19 according to the invention. Clamping means 19 can take any suitable form which enables opposing ends 11 and 12 to be maintained in close proximity or in contact.

In other preferred embodiments, at least one opposing end 11 or 12 includes a tail member 11A or 12A. Tail member 11A or 12A may be substantially linear or . substantially curved and may have a tail surface (not shown) which corresponds with at least a clamping means surface (not shown) on the clamping means 19. In other preferred embodiments, tail members 11A and/or 12A may themselves inter-engage thereby maintaining opposing ends 11 and 12 in close proximity or in contact. As is well illustrated in FIG. 4C, for example, opposing ends 11 and 12 are maintaining close proximity or in contact when tail member 11A is caused to inter-engage with tail member 12A.

The body section 15 of preferred embodiments of support member 10 may be solid or may have weaker or weakened portions or perforations 60. In some preferred embodiments, the body section 15 has a cross-hatched pattern or is mesh-like and may be formed by a plurality of intersecting strips or substantially linear segments, with weaker or weakened portions or perforations 60 interposed between the intersecting strips or substantially linear portions. The weaker or weakened portions or perforations 60 may be any shape and, in embodiments wherein the body section 15 has a cross-hatched pattern or is mesh-like, such as, for example, as depicted in FIGS. 14A and 14D, their shape may be defined by the intersecting strips or substantially linear segments.

As illustrated in FIGS. 16A to 16E, in some preferred embodiments, joining means 71 join two or a plurality of support members 10 together such that their body sections 15 are adjacent and/or substantially parallel to each other. According to some such embodiments, each support member 10 forms a rib-like component of the resultant greater sized support member. The rib-like components of some such embodiments provide the change from the first configuration to the second configuration, while the joining means 71 provide the change to the substantially tubular configuration. As can be appreciated, a support member 10 itself is formed of a plurality of such rib-like components so joined, or two or a plurality of support members 10 are so joined giving rise to the above described “greater sized support member”.

In some such preferred embodiments, the joining means 70 are expandable or extendible, including, for example, by being formed as a spring-like structure which may extend from a compressed state. The ability of the joining means 70 to expand or extend can be particularly useful to minimise the size of support member 10 for loading or preloading into deployment means 20. One example of compression of support member 10 according to one such embodiment is depicted in progressive FIGS. 16B to 16D which illustrate support member 10 in the first configuration being compressed for loading or preloading into deployment means 20 (not shown in these figures).

In some preferred embodiments wherein joining means 70 join two or a plurality of support members 10 together, or wherein a support member 10 is formed of a plurality of rib-like components so joined, the joining means 70 may be formed of material having first shape memory properties and the support members 10 or rib-like components may be formed of another material having second shape memory properties. For example, the first shape memory properties may have a different “time constant” to the second shape memory properties, such that expansion or extension of the joining means 70 (and, therefore, change to the substantially tubular shape configuration) occurs more quickly than the change from the first substantially linear or planar configuration to the second substantially non-linear or prismatic configuration.

The depiction in FIG. 16E illustrates a support member 10 deployed extra-vascularly to support artery 50. Assuming that support member 10 as depicted was formed with at least two different materials, one for the rib-like components and the other for the joining means 70, and those materials had the difference in speed characteristic described above, opposing ends of the rib-like components would have encircled the artery 50 wall more quickly than the joining means 70 would have extended or expanded, thereby causing the support member 10 to extend along the exterior wall of artery 50 as it changed to a substantially tubular configuration.

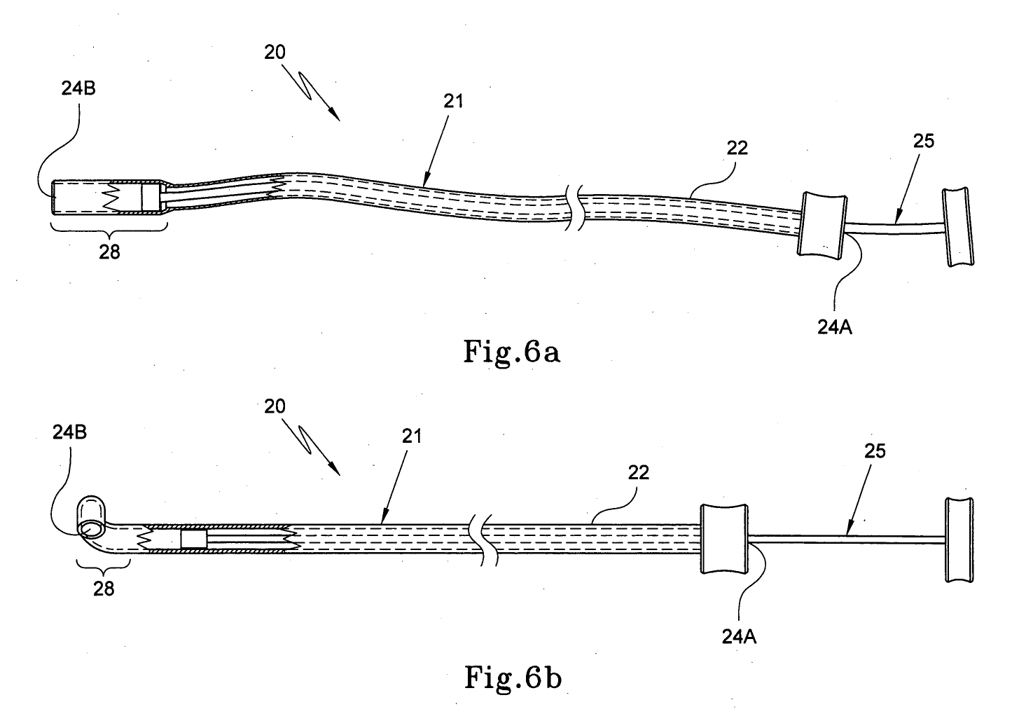

The present invention also provides deployment means 20 adapted for deploying a support member 10. Deployment means 20 comprises a substantially hollow member 21 to deliver support member 10; and an ejection means 25 adapted to aid in the deployment of support member 10 from the substantially hollow member 21. Deployment means 20 is depicted in various embodiments throughout the figures, particularly FIGS. 5A to 7E.

The deployment means 20 depicted in FIG. 5A is particularly well suited for deploying a support member 10 which has a first substantially linear configuration and a second substantially non-linear configuration. The substantially hollow member 25 is defined by substantially tubular wall 22 and has openings 24A and 24B. Ejection means 25 is adapted to be inserted into substantially hollow member 21 and, as illustrated by the two-way arrows, can be advanced or retracted through substantially hollow member 21.

In use, deployment means 20 is advanced into a human or animal body (not shown) or into the vasculature or other vessel system in the human or animal body. The leading end of deployment means 20 is the end adjacent opening 24B. Deployment means 20 is adapted so as to have support member 10 loaded or pre-loaded into substantially hollow member 21 at, or advanced towards, the leading end. Ejection means 25 is adapted to the be loaded or pre-loaded into the substantially hollow member 21 proximal support member 10.

Deployment means 20 is depicted in FIG. 5A as adapted so that once the leading end of deployment means 20 is adjacent a preferred deployment site, support member 10 is deployed by advance of ejection means 25 relative to substantially hollow member 21, or retraction of substantially hollow member 21 relative to ejection means 25.

Deployment means 20 depicted in FIG. 5B is particularly well adapted for deploying a support member 10 which has a first configuration which is either substantially linear or substantially non-linear and a second configuration which is substantially non-linear. In this embodiment, substantially hollow member 21 is defined by substantially tubular wall 22 with openings 24A and 24B. An aperture 23 is formed along a portion of substantially tubular wall 22 extending from and forming part of opening 24B.

Ejection means 25 is also formed of a substantially tubular wall having an opening shown as adjacent (due to its position as depicted) opening 24B of substantially hollow member 21, and an aperture in the substantially tubular wall depicted adjacent (again due to its position as depicted) aperture 23 in the substantially hollow member 21. In this embodiment ejection means 25 is preferably formed of a firm but flexible material so as to enable the ejection means 25 to be not only advanced or retracted relative to substantially hollow member 21, but to also be rotatable relative to substantially hollow member 21, even when the curved trailing end of ejection means 25 is within the curved trailing end of substantially. hollow member 21.

In this embodiment, ejection means 25 is rotated relative to substantially hollow member 21 so that aperture 23 is effectively “closed”. Support member 10 is then loaded or pre-loaded into or adjacent overlapping portions of ejection means 25 and substantially hollow member 21 particularly in the vicinity of aperture 23. When the leading end of deployment means 20 is adjacent the preferred deployment site, ejection means 25 is rotated relative to substantially hollow member 21 so as to effectively “open” aperture 23 enabling deployment of support member 10. Ejection means 25 may further be retracted to aid in the deployment of support member 10.

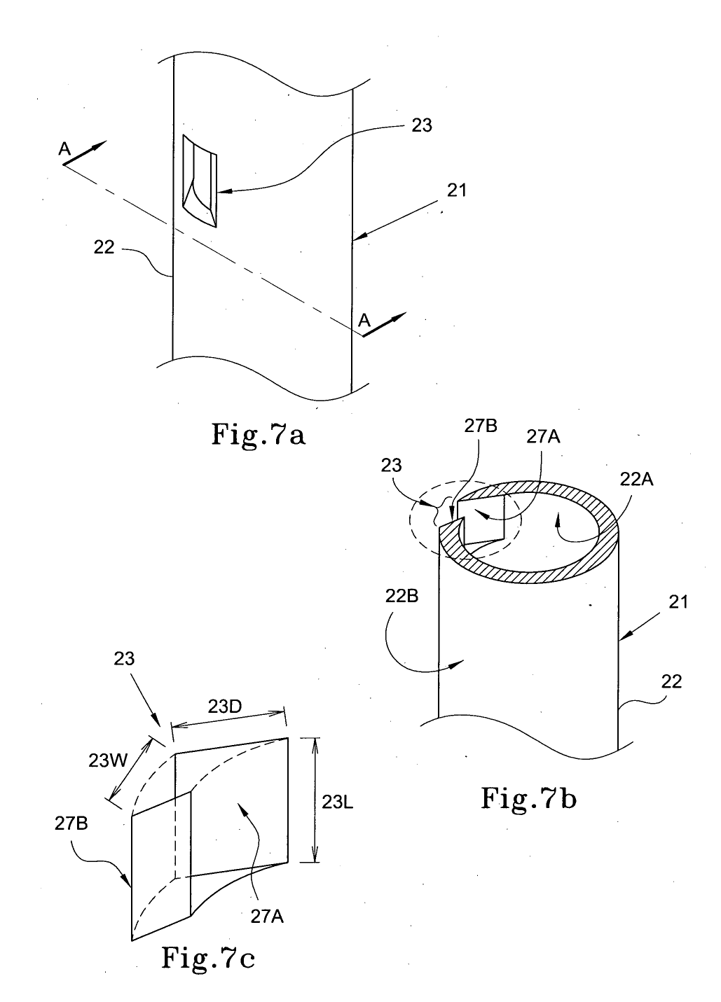

FIG. 6A depicts a yet further preferred embodiment of deployment means 20 according to the present invention. This embodiment of deployment means 20 is particularly well suited for deploying a support member 10 having a first substantially linear or substantially non-linear configuration and a second substantially non-linear configuration.

Substantially hollow member 21 is defined by substantially tubular wall 22 and has openings 24A and 24B. Opening 24b is in the vicinity of the leading end of deployment means 20. As illustrated in this embodiment, region 28 in the vicinity of the leading end of deployment means 20 is specifically adapted to accommodate support member 10 and a leading end of ejection means 25 in this particular embodiment. The adaptation in this case is an increase in cross-sectional diameter of substantially hollow member 21. Ejection means 25 is loaded or pre-loaded into substantially hollow member 21 and is adapted so as to be able to advanced relative to substantially hollow member 21 (or so as to have substantially hollow member 21 retracted) a distance at least equivalent to a length of region 28 in this embodiment. When the leading end of deployment means 20 is adjacent the preferred deployment site, ejection means 25 is advanced relative to substantially hollow member 21 or substantially hollow member 21 is retracted relative to ejection means 25 thereby deploying support member 10 from opening 24B. This particular embodiment is also depicted in FIG. 6A as having considerable flexibility as illustrated by the varying curvatures in substantially tubular wall 22 and in the substantially tubular wall (particularly marked in dotted lines) of ejection means 25 running along internally within substantially hollow member 21.

FIG. 6B depicts yet a further preferred embodiment of deployment means 20 according to the present invention. This embodiment is particularly well suited for deploying a support member 10 wherein the first configuration is substantially linear and the second configuration is substantially non-linear, even more particularly well suited for intravascular deployment of such an embodiment of support member 10.

Substantially hollow member 21 is defined by substantially tubular wall 22 having openings 24A and 24B. Region 28 in the vicinity of the leading end of deployment means 20 is formed, or can be manipulated, so that a distal end curves so as to advance substantially perpendicularly to a longitudinal axis of substantially hollow member 21. Preferably, the distal end also contours a portion of the vessel wall. Ejection means 25 is adapted to be loaded or pre-loaded into substantially hollow member proximal support member 10.

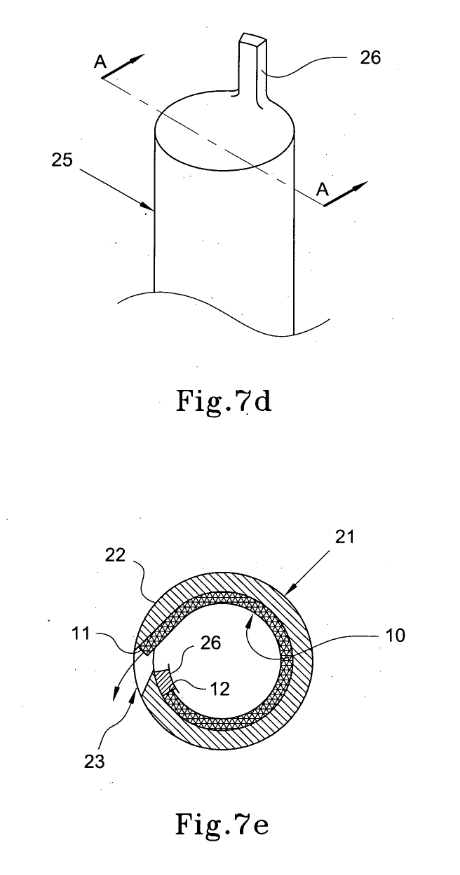

When the leading end of deployment means 20 is adjacent a preferred deployment site, or when its curved distal end is in the same or similar circumferential plane as the deployment site, ejection means 25 is advanced relative to substantially hollow member 21 (or substantially hollow member 21 is retracted relative to ejection means 25) until support member 10 is deployed through region 28 out opening 24B. In this way, support member 10 can be deployed substantially perpendicular to a longitudinal axis of the vessel 50 in which it is being deployed. FIGS. 7A-7E depict various components according to a further preferred embodiment of deployment means 20. This embodiment of deployment means 20 is particularly well adapted for deploying a support member 10 having a first configuration which is substantially non-linear or substantially prismatic with a curved portion or region, and a second configuration which is substantially non-linear or substantially prismatic with a curved portion or region.

FIG. 7A is a cut-off perspective view of a substantially hollow member 21 adapted for this embodiment of deployment means 20. Substantially hollow member 21 is defined by substantially tubular wall 22. Aperture 23, providing fluid communication between at least a portion of the hollow and a space outside substantially hollow member 21 extends through substantially tubular wall 22. As shown in FIG. 7B, which is representative of a cross-sectional view along line A-A in FIG. 7A, aperture 23 extends through the full thickness of substantially tubular wall 22 from inner surface 22A to outer surface 22B.

In preferred embodiments, aperture 23 has a first aperture width adjacent one side of the aperture depth (that is, where aperture 23 penetrates outer surface 22B of substantially tubular wall 22). In the embodiment depicted in FIG. 7B, the first aperture width is smaller than the second aperture width. However, in other embodiments, the first aperture width may be equal to or greater than the second aperture width.

FIG. 7C provides a cut off perspective view of aperture 23 as circled in FIG. 7B. This illustrates aperture 23 having an aperture width 23W, aperture depth 23D and aperture length 23L. Aperture 23 is defined on opposing sides along the aperture length 23L by first and second aperture side walls 27A and 27B. Each aperture side wall 27A and 27B have an aperture side wall axis.

In the particularly preferred embodiment depicted in FIGS. 7B and 7C, the aperture side wall axes are substantially un-parallel. Preferably, the first aperture side wall 27A extends tangentially from inner surface 22A of the substantially tubular wall 22 until it meets outer surface 22B of substantially tubular wall 22. The second aperture side wall 27B also extends from inner surface 22A at an acute angle to inner surface 22A in a direction away from first aperture side wall 27A until it meets outer surface 22B of the substantially tubular wall 22.

In the preferred embodiment depicted in FIGS. 7A, 7B and 7C, aperture length 23L is greater than aperture width 23W and/or aperture depth 23D. Preferably, aperture length 23L, aperture depth 23D and aperture width 23W are of a size adapted to at least accommodate a support member 10, or a cross-, longitudinal-, or other-, section of support member 10, passing through aperture 23. As depicted in FIGS. 7A, 7B and 7C, a longitudinal axis of aperture length 23L is substantially parallel to a longitudinal axis of substantially tubular wall 22. In alternative embodiments, the longitudinal axis of aperture length 23L may be unparallel to the longitudinal axis of substantially tubular wall 22, or may have other such physical relationships to the longitudinal axis of tubular wall 22.

Ejection means 25 of a preferred embodiment adapted for deployment means 20 of the embodiment depicted in FIGS. 7A-7E is illustrated in perspective view in FIG. 7B. As can be seen, ejection means 25 is an elongate member adapted to be inserted into the substantially hollow member 21. Ejection means 25 of this embodiment further includes guide means 26 in the form of a guide member extending from a leading end of the elongate member. In the embodiment illustrated in FIG. 7D, the guide member 26 extends from an outer edge of the elongate member of ejection means 25.

As is well illustrated in FIG. 7E (which is a cross-section of deployment means 20 through line A-A of FIGS. 7A and 7D), at least a portion of guide member 26 is adapted to locate between opposing ends 11 and 12 of support member 10. As illustrated in FIG. 7E, because guide member 26 locates between opposing ends 11 and 13 of support member 10, in cross-section, the guide member 26 effectively “fills a gap” between opposing ends 11 and 12 of the support member, or at least “adds” to the incomplete circle provided by support member 10 in the first substantially non-linear or substantially prismatic, with a curved portion or region, configuration of support member 10. In some such embodiments, a side of guide member 26 can abut one opposing end 11 or 12 of support member 10. Support member 10 can then be deployed through aperture 23 in substantially tubular wall 22 by relative rotation between ejection means 25 and substantially hollow member 21.

In some embodiments, guide means 26 may further include additional means or members adapted to further aid deployment of support member 10 through aperture 23. For example, in one embodiment, guide means 26 further includes outward pressure applying means (not shown) adapting to provide an outward pressure to opposing end 11 or 12 of support member 10 which leads deployment of support member 10 through aperture 23.

In some further preferred embodiments, a trailing end or other suitable part of support member 10 is joined to ejection means 25 or guide means 26 by any one of a number of joins including frangible joins, snap-off joins, tearible joins, breakable joins, detachable joins and a combination of two or more thereof. In some preferred embodiments, the join is an electrolytically detachable join adapted so that when an electric current is passed through to ejection means 25 and/or support member 10, the electrolytic join is broken, releasing support member 10.

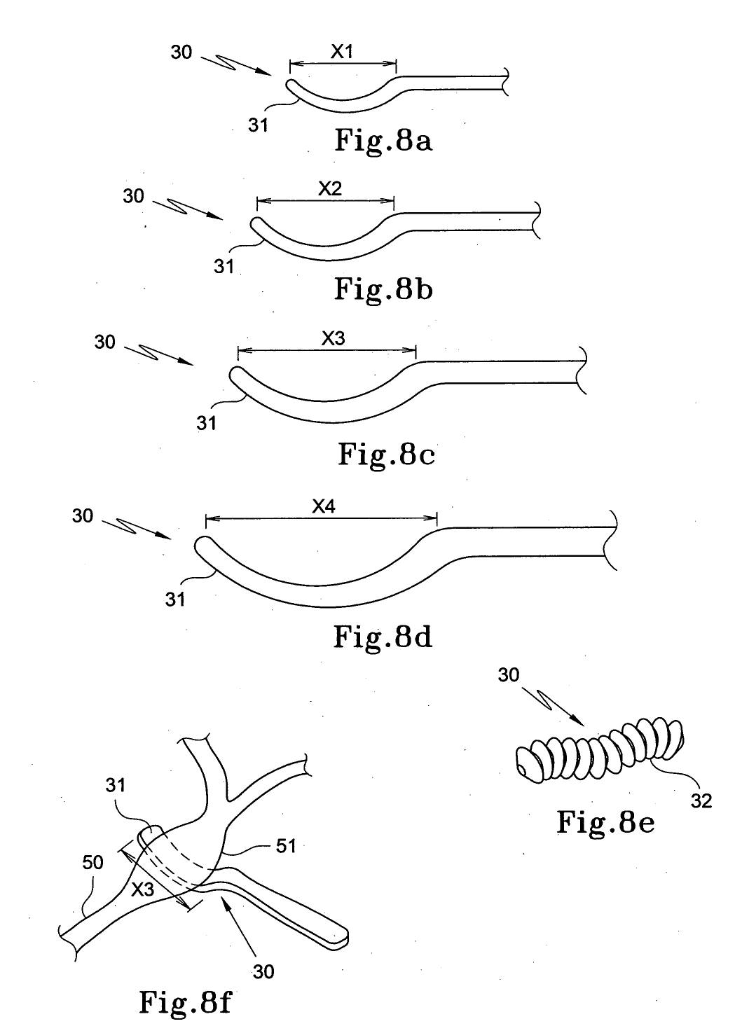

Preferred embodiments of the present invention also provide measuring means 30 in the form, preferably, of a probe-like structure adapted to aid in determination of requisite support member geometry for a particular surgical circumstance. Various embodiments of suitable measuring means 30 according to the invention are depicted in FIGS. 8A to 8F. In some preferred embodiments, a plurality of probe-like structures with substantially fixed physical characteristics 31 are advanced adjacent the deployment site one by one to assist in determining requisite support member geometry. Measuring means 30, as depicted in FIGS. 8A to 8D and 8F have substantially fixed characteristics 31 with an arc of curvature having a shape adapted to generally correspond to the shape of an arc of curvature on an inner or outer part of a vessel wall and/or an inner or outer part of an abnormality in a vessel wall, such as a stenosis 52 or an aneurysm 51.

Measuring means 30 depicted in FIGS. 8A to 8D show fixed physical characteristics 31 with an arc of curvature having a respective distance of X1 to X4 between a distal portion and a proximal portion of the arc of curvature. As illustrated well in FIG. 8F, measuring means 30 depicted in FIG. 8C having fixed physical characteristics 31 with a distance X3 between distal and proximal ends of the arc of curvature corresponds most closely to an arc of curvature on an outer part of vessel 50 wall and aneurysm 51. Following this determination, measuring means 30 is retracted from adjacent the deployment site and a support member 10 having corresponding geometry to the geometry of substantially fixed characteristics 31 is selected for use.

In another preferred embodiment, a probe-like structure having variable physical characteristics, including, for example, variable lengths, angles and/or terminal curves adapted to generally correspond to certain physical characteristics of a support member 10 in the first, second or other configuration, is advanced into the deployment site. An example of an embodiment of a measuring means 30 suitable for this purpose is depicted in FIG. 8E. In this particular embodiment, variable physical characteristics 32 are provided by a mechanism and/or material capable of behaving in a similar fashion to the flexible component of a bendable drinking straw. A measuring means 30 according to this embodiment is advanced adjacent the deployment site and manipulated until its geometry matches or corresponds to an arc of curvature or a substantial circumference of an inner or outer part of a vessel 50 wall and/or an inner or outer part of an abnormality in a vessel 50 wall, such as a stenosis 52 or an aneurysm 51. The invention envisages a range of mechanisms and/or materials for the measuring means 50 having variable physical characteristics 32.

In this description, various preferred embodiments for deployments means 20 have been described as being particularly well suited or adapted for deploying particular embodiments of support members 10. Persons skilled in the art will, however, appreciate that the description of certain combinations of particular embodiments of deployment means 20 with particular embodiments of support members 10 are exemplary only, and that other combinations may also be suitable.

The present invention also provides a method of supporting a human or animal vessel 50 comprising: deploying a vessel support member 10 according to the present invention at, or in the vicinity of, a deployment site such that the support member 10 substantially contours a portion of a circumference of the vessel 50, thereby supporting the vessel 50. Preferably, the deployment site is within, or adjacent, substantially the same or similar circumferential plane as a portion of vessel 50 which is abnormal, such as a stenosis 52 or an aneurysm 51.

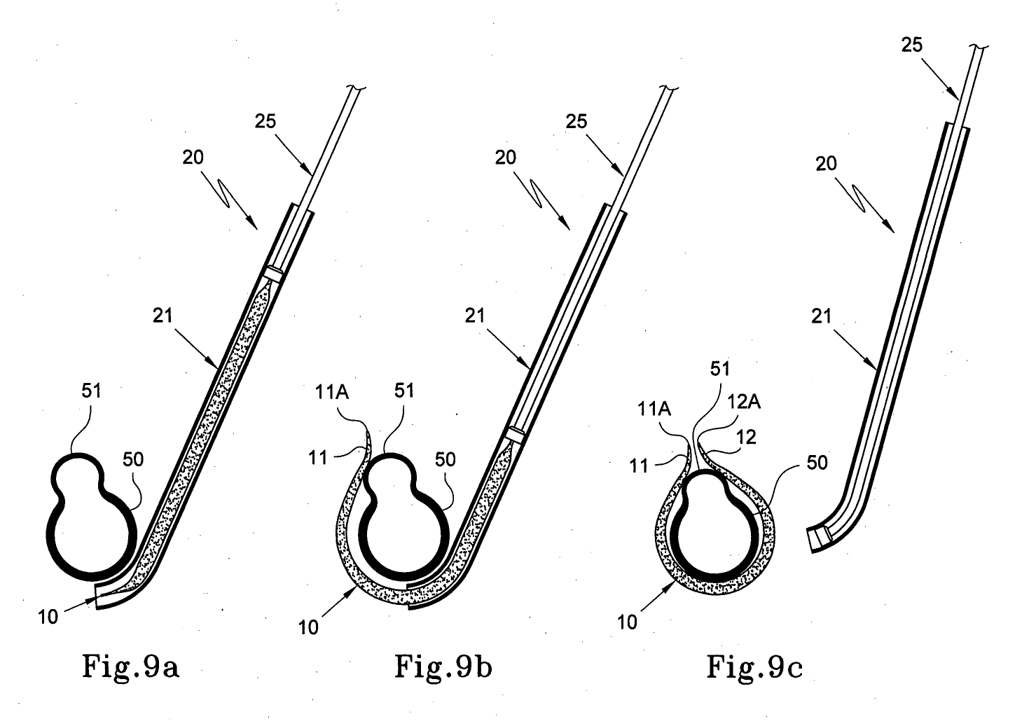

The support member 10 can be deployed according to the method of the present invention either intra-vascularly or extra-vascularly. Typically, to support a vessel 50 with a stenosis 52, the support member 10 is deployed intra-vascularly and to support a vessel 50 with an aneurysm 51, the support member 10 is deployed intra- or extra-vascularly. FIGS. 9A to 9C depict progressive steps in the extra-vascular deployment of a support member 10 to support a vessel 50 with an aneurysm 51.

In FIG. 9A, deployment means 20 is loaded or pre-loaded with support member 10 and ejection means 25 is loaded or pre-loaded proximal to support member 10 in substantially hollow member 21. Deployment means 20 is advanced into the extra-vascular space until adjacent the wall of vessel 50. Once in the vicinity of the deployment site, as shown in FIG. 9B, ejection means 25 is advanced relative to substantially hollow member 21 (in another preferred embodiment, substantially hollow member 21 could be retracted relative to ejection means 25 with the same result) and support member 10 is deployed, preferably until opposing end 11 with or without first tail member 11A is adjacent or abuts one opposing side or wall of aneurysm 51.

As shown in FIG. 9C, ejection means 25 is made to continue advancing until support member 10 has been fully deployed and has changed into the second substantially non-linear configuration. Preferably, support member 10 substantially encircles the vessel and the aneurysm as depicted well in FIG. 9C. The deployment means 20 can then be retracted out of the deployment site and out of the human or animal body.

Once fully deployed, opposing ends 11 and 12 (with or without first and second tail members 11A and 11 B) are adjacent or abut opposing sides or walls of aneurysm 51. In some such preferred embodiments, when opposing ends 11 and 12 come into close proximity to each other or into contact, the aneurysm is compressed between the opposing sides and maybe substantially obliterated or substantially eliminated.

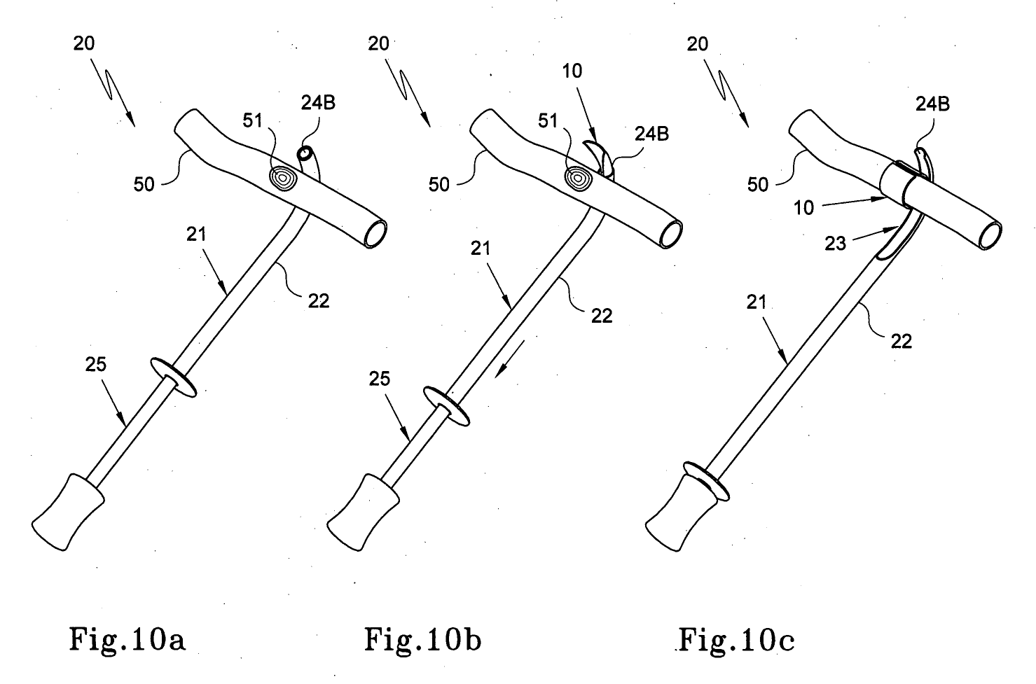

FIGS. 10A and 10B show a perspective view of similar embodiments of deployment means 20, vessel 50 and aneurysm 51 as were depicted in FIGS. 9A to 9C. As can be seen by the differences between FIGS. 10A and 10B, as ejection means 25 is advanced into substantially hollow member 21 it pushes support member 10 out through aperture 24B. This is shown in FIG. 10B where support member 10 protrudes from opening 24B.

FIG. 10C illustrates a perspective view of the deployment means depicted in FIG. 5B having been used to deploy support member 10 to support vessel 50. Ejection means 25 is not referenced in this diagram as it has been fully inserted into substantially hollow member 21. As can be seen, aperture 23 forms part of and extends from opening 24B in substantially tubular wall 22. The embodiment of deployment means 20 in FIG. 10C is different to the deployment means 20 depicted in FIGS. 10A and 10B for illustrative purposes. However, as illustrated in FIG. 10C once deployed, support member 10 substantially contours an outer surface of vessel 50 and aneurysm 51 thereby supporting vessel 50. This end result of support member 10 having been deployed to support vessel 50 is the preferred result whichever deployment means is used.

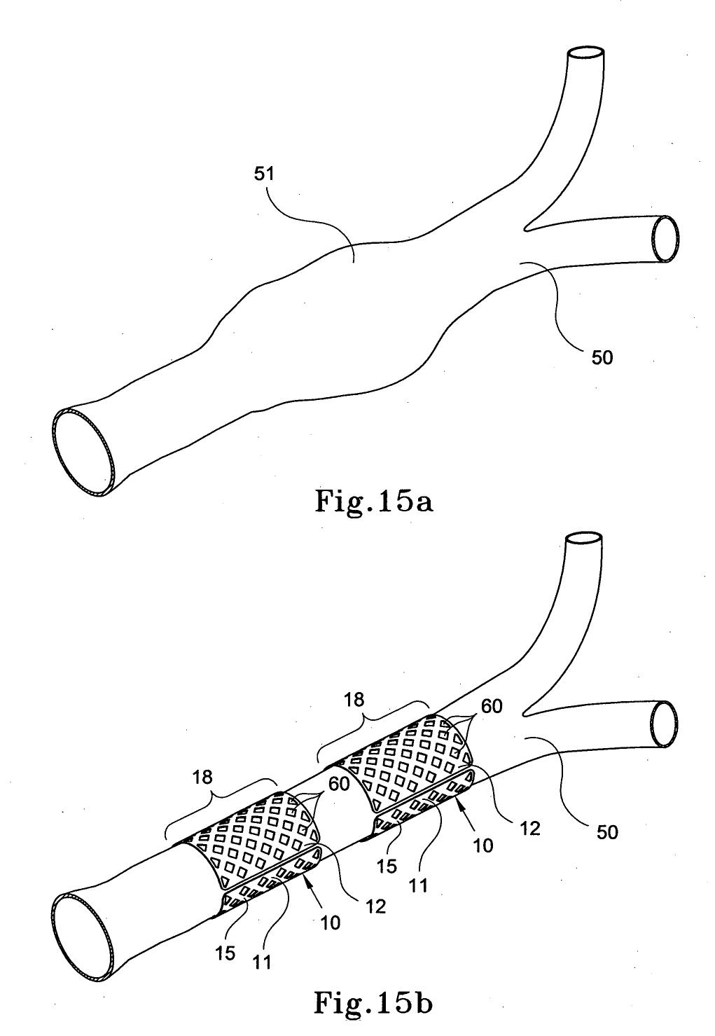

FIGS. 14B and 14C depict sequential schematic representations of one preferred embodiment of support member 10 (as shown in FIG. 14A) being deployed from deployment means 20. As can be seen, as ejection means 25 is relatively advance through substantially hollow member 21, support member 10 changes from the first configuration to at least the second configuration. As depicted in FIGS. 15A and 15B, in some circumstances, it may be desirable to deploy more than one support member 10 to support a vessel 50 and, for example, an aneurysm 51. In FIG. 15B, two support members 10 of a preferred embodiment (as shown in FIG. 14A) are depicted as having been deployed adjacent one another in order to support vessel 50 and to support and contain aneurysm 51.

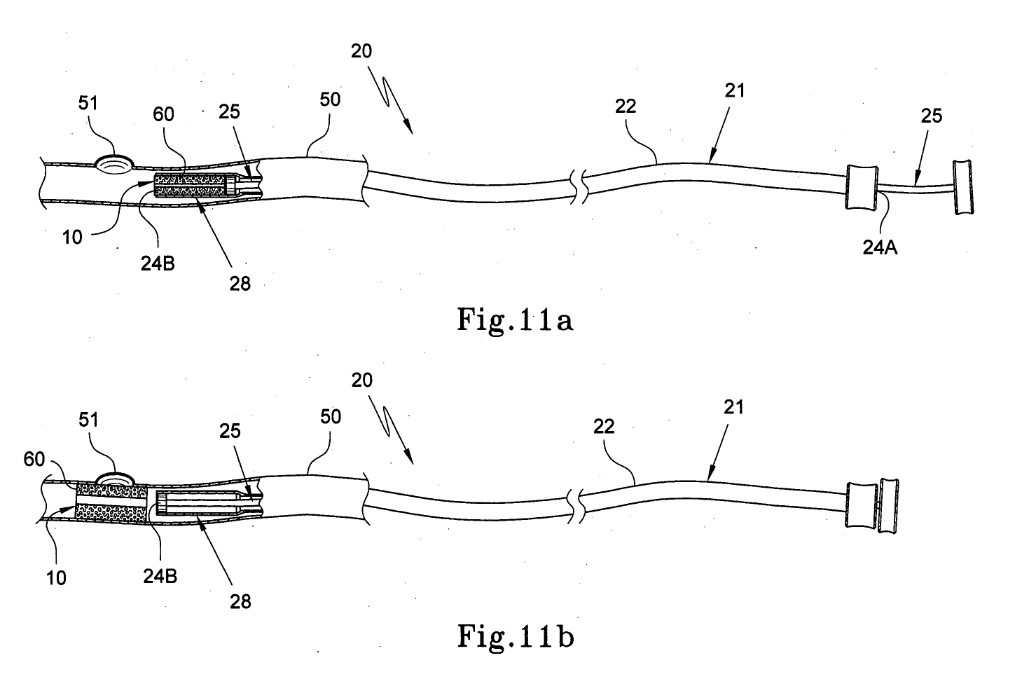

FIGS. 11A through 13C focus on intra-vascular deployment. FIGS. 11A and 11B depict sequential schematic diagrams of a method of deploying a support member 10 intra-vascularly to support a vessel 50 having an aneurysm 51. In this embodiment, region 28 of substantially hollow member 21 has a slightly larger diameter so as to accommodate leading end of ejection means 25 and support member 10 in the first configuration. Deployment means 20 loaded or pre-loaded with support member 10 and ejection means 25 is advanced along the vasculature into vessel 50 until opening 24B of substantially hollow member 21 is adjacent a preferred deployment site. Ejection means 25 is then advanced along substantially hollow member 21 until support member 10 is fully deployed as shown in FIG. 11B. Support member 10 then substantially contours an inner wall of vessel 50 across neck of aneurysm 51.

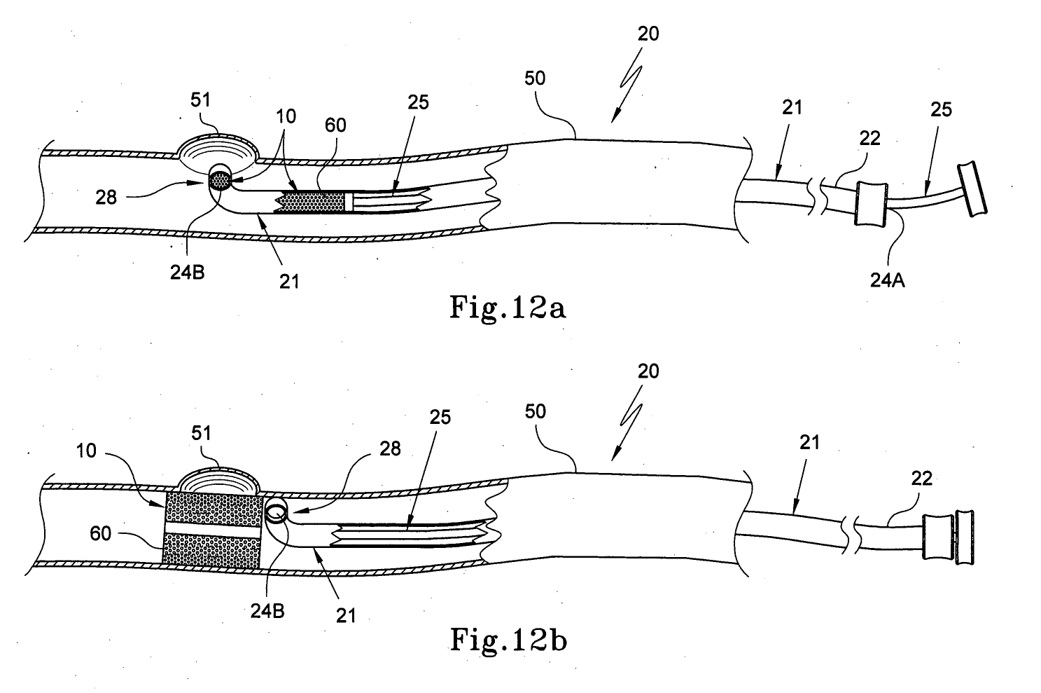

FIGS. 12A and 12B illustrate sequential steps in deployment of support member 10 intra-vascularly to support a vessel 50 having an aneurysm 51. In this embodiment of the method, deployment means 20 depicted in FIG. 6B is illustrated as being used. As can be seen, deployment means 20 is advanced until region 28 of substantially hollow member 21 is in the same or similar circumferential plane as aneurysm 51. Once positioned as such, ejection means 25 is advanced along substantially hollow member 21 until support member is deployed out of opening 24B.

As shown in FIG. 12B, support member 10 has been fully deployed so as to contour an inner wall of vessel 50 and span neck of aneurysm 51. Accordingly, in this embodiment of the method, support member 10 in a first substantially linear configuration is loaded or pre-loaded into substantially hollow member 21. Although not shown, in the first instance support member 10 deploys from opening 24B and as it deploys, changes into the second substantially non-linear configuration, which is substantially C-shaped or substantially circular. Support member 10 then undergoes transverse extension 18 until it reaches a third configuration which is substantially tubular. An example of an embodiment of a support member 10 which deploys as such is illustrated in FIG. 2.

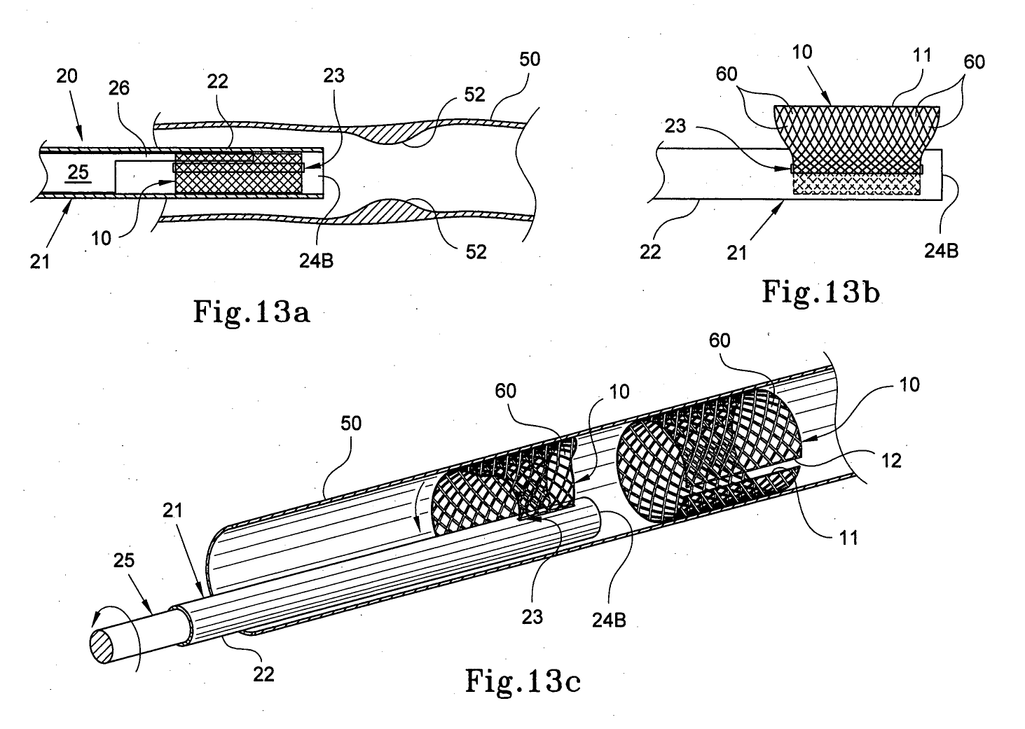

FIG. 13A depicts a vessel 50 having a stenosis 52. Deployment means 20 as embodied according to the illustrations in FIGS. 7A to 7E is shown advancing in vessel 50 towards stenosis 52. In this embodiment, support member 10 is loaded or pre-loaded into substantially elongate member 21 in a first substantially non-linear or substantially prismatic shape with a curved portion or region in the vicinity of aperture 23. Ejection means 25 is then loaded or pre-loaded into substantially hollow member 21 so that at least part of guide member 26 locates between opposing ends 11 and 12 of support member 10.

As illustrated in FIG. 13B, relative rotation of ejection means 25 (not shown) with respect to substantially hollow member 21 results in deployment of one opposing end 11 of support member 10 through aperture 23. Once fully deployed, as shown in FIG. 13C, support member 10 contours a substantial portion of vessel 50 forcing stenosis (not shown) to expand or, in any event, for vessel to re-establish patency of similar magnitude to that in the vessel before the stenotic lesion occurred. FIG. 13C also illustrates that more than one support member 10 can be deployed to support vessel 50 in appropriate circumstances.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A vessel support member having opposing ends adapted to move in a circumferential plane, the support member configured for deployment in a substantially radial direction to support a substantial portion of the circumference of a vessel,
the support member adapted to change between a first configuration and at least a second configuration, such that when in the first configuration, the support member is substantially linear or substantially planar, and as the support member is deployed and changes to at least the second configuration, one opposing end moves in a radial 10 direction relative to the other opposing end, such that when in the second configuration the support member is substantially non-linear or substantially prismatic with a curved portion or region.

2. The vessel support member of claim 1, adapted to support a substantial portion of,
or the whole of, a circumference of the vessel, and any variation to the circumference of the vessel, whether from adjacent an inner wall or an external wall of the vessel.

3. The vessel support member of claim 1, wherein a cross-section of the support member in the second configuration has a substantially C-shape or is substantially circular.

4. The vessel support member of claim 1, adapted to further change
to a third configuration, such that the support member has a substantially tubular shape.

5. The vessel support member of claim 1, comprising two or a plurality of rib-like components, with each rib-like component having opposing ends adapted to move in a circumferential plane and a body section interposed between the opposing ends, and joining means adapted to join the two or plurality of rib-like components.

6. The vessel support member of claim 5, wherein the rib-like components provide change from the first configuration to the second configuration, and the joining means provide change to a third configuration.

7. The vessel support member of claim 4, further adapted for preferential regulation of change between the first and second configurations and change to the third configuration.

8. The vessel support member of claim 7, wherein a rate of change from the first configuration to the second configuration and a rate of change to the third configuration are different to one another.

9. The vessel support member of claim 1, formed of at least one material having shape memory properties and a malleable or cloth-like material having biocompatible properties connected or secured to the material having shape memory properties.

10. The vessel support member of claim 1, wherein the opposing ends are maintained in close proximity or in contact in the second, third or further configuration.

11. The vessel support member of claim 10, wherein the opposing ends are maintained in close proximity or in contact by clamping means.

12. Deployment means adapted for deploying a support member according to claim 1, the deployment means comprising:
a substantially hollow member adapted to deliver the support member; and
an ejection means adapted to aid in the deployment of the support member from the substantially hollow member.

13. The deployment means of claim 12, wherein a trailing end the support member is joined to the ejection means, by a join selected from the group consisting of a frangible join, a snap-off join, a tearable join, a breakable join, a detachable join, an electrolytically detachable join, and a combination of two or more thereof.

14. A kit for providing support to a vessel in a human or animal body, the kit comprising:
a support member according to claim 1; and
a deployment means comprising:
a substantially hollow member adapted to deliver the support member; and
an ejection means adapted to aid in the deployment of the support member from the substantially hollow member.

15. The kit of claim 14 further including at least one probe-like structure adapted to assist in determining requisite support member geometry.

16. A method of supporting a human or animal vessel comprising:
deploying a vessel support member according to claim 1 at, or in the vicinity of, a deployment site such that the support member substantially contours a portion of a circumference of the vessel, thereby supporting the vessel.

17. The method of claim 16 wherein the deployment site is within, or adjacent, substantially the same or similar circumferential plane as a portion of the vessel which is abnormal.

18. The method of claim 18, wherein the vessel is an artery and the support member is deployed intra-vascularly or extra-vascularly or one or a plurality of support members is deployed intra-vascularly and one or a plurality of support members is deployed extra-vascularly.

19. The method of claim 16, wherein the support member, in the first configuration, is loaded or pre-loaded into deployment means, and as the support member is deployed, the support member changes from the first configuration to at least the second configuration.

20. The method of claim 16 wherein a determination of requisite support member geometry is made in an initial step, using a probe-like structure having variable physical characteristics adapted to correspond to relevantly corresponding physical characteristics of the support member, or using a plurality of probe-like structures with substantially fixed physical characteristics adapted to correspond to relevantly corresponding physical characteristics of support members of varying physical characteristics.
